(19) United States
(12) Patent Application Publication
(10) Pub. No.: US 2006/0031961 A1
(43) Pub. Date: Feb. 9, 2006

(54) Aldehyde reductase

(76) Inventors: Brian McGonigle, Wilmington, DE (US); John D. Everard, Wilmington, DE (US); David G. Nekoukar, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/198,728

(22) Filed: Aug. 5, 2005

Related U.S. Application Data
(60) Provisional application No. 60/599,042, filed on Aug. 5, 2004.

(51) Int. Cl. A01H 5/00; C12N 5/04; C07H 21/04; C12N 9/02; C12N 15/82; A61K 36/48

(52) U.S. Cl. 800/278; 424/757; 800/312; 435/189; 435/415; 435/468; 536/23.2

(57) ABSTRACT

This invention relates to an isolated polynucleotide encoding an aldehyde reductase enzyme. The invention also relates to the construction of a recombinant construct encoding an aldehyde reductase enzyme wherein expression of the recombinant construct results in production of altered levels of aldehyde reductase enzyme in a transformed host cell. The present invention also concerns soybean products made from seeds obtained from soybean plants transformed with this recombinant construct wherein such seeds having increased aldehyde reductase activity.

This application claims the benefit of U.S. Provisional Application No. 60/599,042, filed Aug. 5, 2004, the entire content of which is herein incorporated by reference.

### FIELD OF THE INVENTION

This invention is in the field of plant molecular biology. This invention relates to an isolated polynucleotide encoding an aldehyde reductase. More specifically, this invention pertains to polynucleotides useful in increasing aldehyde reductase activity in soybeans. Included in the invention are soybean seeds having increased aldehyde reductase activity.

### BACKGROUND OF THE INVENTION

Soybean-based food products have “beany” and “grassy” off-flavors that limit the potential for wider use of this economical and healthy source of protein. A great deal of research has been undertaken to understand the source of these off-flavors. Evidence indicates that aldehydes and ketones contribute to the production of these off-flavors.

Aldehyde reductase, (E.C. 1.1.1.2), is a member of the aldo-keto reductase superfamily. It falls into a group of enzymes classified as oxidoreductases. Aldehyde reductase converts aldehydes or ketones to their respective alcohols. Accordingly, the availability of nucleic acid sequences encoding all or a portion of these enzymes would facilitate studies to better understand aldo/keto metabolism and function in plants, provide genetic tools for the manipulation of the aldo/keto levels, and provide a means to improve the flavor of soy protein products by controlling the accumulation of aldehydes and ketones.

U.S. Pat. No. 6,274,379 B1, issued to Famodu et al. on Aug. 14, 2001, concerns isolated nucleic acid fragments encoding sorbitol biosynthetic enzymes, specifically, nucleic acid fragments encoding an aldehyde reductase, NADP-dependent D-sorbitol-6-phosphate dehydrogenase or sorbitol dehydrogenase. The focus appears to be carbohydrate metabolism and a means to control carbon partitioning in plant cells.

U.S. Pat. No. 4,232,044, issued to Chiba et al. on Nov. 4, 1980, describes the use of enzymes exogenously added during processing to improve protein flavor. In particular, the use of aldehyde dehydrogenase, aldehyde oxidase and alcohol dehydrogenase to improve the flavor of proteins containing aldehydes and/or alcohols is described.

Bartels, Trends in Plant Science, 6(7):284-286 (2001), discusses pathways that can be used to obtain stress-tolerant plants. A stress-activated aldose-aldehyde reductase is described that when expressed in tobacco resulted in tolerance to oxidative stress and dehydration.

Oberschall et al., The Plant Journal 24(4):437-446 (2000), describes the isolation and characterization of an alfafa aldose/aldehyde reductase gene and the encoded enzyme. Tobacco plants that over-produced this alfafa enzyme showed lower concentrations of reactive aldehydes and tolerance against oxidative agents and drought stress.

### SUMMARY OF THE INVENTION

The present invention concerns isolated polynucleotides comprising a nucleotide sequence encoding a polypeptide having aldehyde reductase activity, wherein the amino acid sequence of the polypeptide and the amino acid sequence of SEQ ID NO:10 have at least 86% sequence identity. It is preferred that the identity be at least 90%, it is more preferred that the identity be at least 95%. The present invention also relates to isolated polynucleotides comprising the complement of the nucleotide sequence. More specifically, the present invention concerns isolated polynucleotides encoding the polypeptide sequence of SEQ ID NO:10.

In a first embodiment, the present invention includes an isolated polynucleotide comprising: (a) a nucleotide sequence encoding a polypeptide having aldehyde reductase activity, wherein the polypeptide has an amino acid sequence of at least 86%, 90% or 95% sequence identity, based on the Clustal V method of alignment, when compared to one of SEQ ID NO:10, or (b) a complement of the nucleotide sequence, wherein the complement and the nucleotide sequence consist of the same number of nucleotides and are 100% complementary. The polypeptide preferably comprises the amino acid sequence of SEQ ID NO:10. The nucleotide sequence preferably comprises the nucleotide sequence of SEQ ID NO:9.

In a second embodiment, the present invention concerns a recombinant DNA construct comprising any of the isolated polynucleotides of the present invention operably linked to at least one regulatory sequence, and a cell, a plant, and a seed comprising the recombinant DNA construct.

In a third embodiment, the present invention includes a vector comprising any of the isolated polynucleotides of the present invention.

In a fourth embodiment, the present invention concerns a method for transforming a cell comprising transforming a cell with any of the isolated polynucleotides of the present invention. The cell transformed by this method is also included. Advantageously, the cell is eukaryotic, e.g., a yeast or plant cell, or prokaryotic, e.g., a bacterium.

In a fifth embodiment, the present invention includes a method for producing a transgenic plant comprising transforming a plant cell with any of the isolated polynucleotides of the present invention and regenerating a plant from the transformed plant cell. The invention is also directed to the transgenic plant produced by this method, and seed obtained from this transgenic plant.

In a sixth embodiment, the present invention concerns an isolated polypeptide having aldehyde reductase activity, wherein the polypeptide has an amino acid sequence of at least 86%, 90% or 95% identity, based on the Clustal V method of alignment, when compared to one of SEQ ID NO:10. The polypeptide preferably comprises the amino acid sequence of SEQ ID NO:10.

In a seventh embodiment, the present invention includes a method for isolating a polypeptide having aldehyde reductase activity comprising isolating the polypeptide from a cell or culture medium of the cell, wherein the cell comprises a recombinant DNA construct comprising a polynucleotide of the invention operably linked to at least one regulatory sequence.

In a eighth embodiment, this invention concerns a method of altering the level of expression of an aldehyde reductase protein in a host cell comprising: (a) transforming a host cell with a recombinant DNA construct of the present invention; and (b) growing the transformed host cell under conditions that are suitable for expression of the recombinant DNA construct wherein expression of the recombinant DNA construct results in production of altered levels of the aldehyde reductase protein in the transformed host cell.

In a ninth embodiment, this invention concerns a soybean product obtained from a soybean seed having increased aldehyde reductase activity, the soybean being produced by a soybean plant comprising in its genome a recombinant construct comprising a nucleotide sequence encoding a polypeptide having aldehyde reductase activity and said nucleotide sequence is operably linked to at least one regulatory sequence. More specifically, the nucleotide sequence encoding a polypeptide having aldehyde reductase activity comprises a nucleotide sequence selected from the group consisting of:

a) a nucleotide sequence encoding a polypeptide having aldehyde reductase activity, wherein the polypeptide has at least 86% sequence identity, based on the Clustal V method of alignment, when compared to SEQ ID NO:10;

b) a nucleotide sequence encoding a polypeptide having aldehyde reductase activity, wherein the polypeptide has at least 95% sequence identity, based on the Clustal V method of alignment, when compared to one of SEQ ID NO:2, SEQ ID NO:4, SEQ ID NO:6 or SEQ ID NO:8;

c) a nucleotide sequence encoding the amino acid sequence of SEQ ID NO:2, SEQ ID NO:4, SEQ ID NO:6, SEQ ID NO:8 or SEQ ID NO:10; and

d) a nucleotide sequence of SEQ ID NO:1, SEQ ID NO:3, SEQ ID NO:5, SEQ ID NO:7 or SEQ ID NO:9.

In a tenth embodiment, this invention concerns foods, beverages and feed incorporating such soybean products.

In an eleventh embodiment, this invention concerns a method for increasing aldehyde reductase activity in soybean seeds by transforming a plant part or plant tissue with a recombinant construct comprising one of the nucleotide sequences of the invention operably linked to at least one regulatory sequence, regenerating a transgenic plant from the transformed plant part or plant tissue and growing the transformed plant under conditions suitable for expression of the construct wherein expression of the construct results in increase aldehyde reductase activity.

In a twelfth embodiment, this invention concerns a method for producing a soy product from the seeds of the invention.

Also included in the invention are the grains from the transgenic plants of the invention. Soybean protein product prepared from grain is also an embodiment of the invention. Oil, feed, food, and industrial products are also contemplated by the present invention.

### BIOLOGICAL DEPOSITS

The following plasmids have been deposited with the American Type Culture Collection (ATCC), 10801 University Boulevard, Manassas, Va. 20110-2209, and bear the following designations, accession numbers and dates of deposit.

| | ATCC Accession | |
|---|---|---|
| Plasmid | Number | Date of Deposit |
| pKS231 | PTA-6148 | Aug. 4, 2004 |

### FIGURES AND SEQUENCE LISTINGS

The invention can be more fully understood from the following detailed description and the accompanying Sequence Listing which form a part of this application.

FIGS. 1A, 1B and 1C show a comparison of the following: (a) the amino acid sequence of the aldehyde reductase encoded by the nucleotide sequence derived from soybean clone sr1.pk0003.c5:fis (SEQ ID NO:6), (b) the amino acid sequence of the aldehyde reductase encoded by the nucleotide sequence derived from soybean clone sls2c.pk027.a23:fis (SEQ ID NO:10), (c) the amino acid sequence of abscisic acid activated (orf) protein from Medicago sativa (NCBI General Identifier No. 3378650 (SEQ ID NO:17)), (d) the amino acid from Glycine max (NCBI General Identifier No. 16239835 (SEQ ID NO:18)) and (e) the amino acid sequence of abscisic acid activated protein from Medicago sativa (NCBI General Identifier No. 7431112 (SEQ ID NO:19)).

FIG. 2 shows a schematic of KS210.

FIG. 3 shows a schematic of pKS231 (ATCC Accession No. PTA-6148).

FIG. 4 shows a schematic of pTASTE19 (PHP20765).

FIG. 5 shows a schematic of pTASTE20 (PHP20766).

Dashes are used by the program to maximize alignment of the sequences.

The sequence descriptions and Sequence Listing attached hereto comply with the rules governing nucleotide and/or amino acid sequence disclosures in patent applications as set forth in 37 C.F.R. §1.821-1.825.

Table 1 lists the polypeptides that are described herein, the designation of the cDNA clones that comprise the nucleic acid fragments encoding polypeptides representing all or a substantial portion of these polypeptides, and the corresponding identifier (SEQ ID NO:) as used in the attached Sequence Listing. The sequence descriptions and Sequence Listing attached hereto comply with the rules governing nucleotide and/or amino acid sequence disclosures in patent applications as set forth in 37 C.F.R. §1.821-1.825.

TABLE 1
Aldehyde Reductase

| | | SEQ ID NO: | |
|---|---|---|---|
| | | | (Amino |
| Protein | Clone Designation | (Nucleotide) | Acid) |
| Aldehyde reductase | Contig composed of: | 1 | 2 |
| | ccase-b.pk0023.g2 | | |
| | ceb5.pk0015.g5 | | |
| | ceb5.pk0044.c8 | | |
| | ceb5.pk0049.d10 | | |
| | ceb5.pk0052.a4 | | |
| | ceb5.pk0052.h8 | | |
| | ceb5.pk0062.a4 | | |
| | ceb5.pk0075.e7 | | |
| | ceb5.pk0075.g12 | | |
| | cen6.pk0001.g2 | | |
| Aldehyde reductase | rca1n.pk022.j17 | 3 | 4 |
| Aldehyde reductase | sr1.pk0003.c5:fis | 5 | 6 |
| Aldehyde reductase | wl1n.pk0078.e5 | 7 | 8 |
| Aldehyde reductase | sls2c.pk027.a23:fis | 9 | 10 |

SEQ ID NO:11 is primer 5′ GGT ATT GAG GGT CGC ATG GCC AAA TCA ATC AAA TTC 3′.

SEQ ID NO:12 is primer 5′ AGA GGA GAG TTA GAG CCT CAG AGT TCA CCA TCC CAG 3′.

SEQ ID NO:13 is primer 5′ GGT ATT GAG GGT CGC ATG GCA AAG TTA ATA AAA TTC 3′.

SEQ ID NO:14 is primer 5′ AGA GGA GAG TTA GAG CCT CAG AGT TCA CCA TCC CAA AG 3′.

SEQ ID NO:15 is the amino acid sequence of aldose reductase (aldehyde reductase) from Hordeum vulgare (NCBI General Identifier No. 113595).

SEQ ID NO:16 is the amino acid sequence of aldose reductase from Avena fatua (NCBI General Identifier No. 2130022).

SEQ ID NO:17 is the amino acid sequence of abscisic acid activated (orf) protein from Medicago sativa (NCBI General Identifier No. 3378650 or NCBI General Identifier No. 7431112 (SEQ ID NO:19)). NCBI General Identifier No. 7431112 and NCBI General Identifier No. 3378650 have identical amino acid sequences.

SEQ ID NO:18 is the amino acid sequence of SEQ ID NO:6 in U.S. Pat. No. 6,274,379, granted Aug. 14, 2001, from Glycine max (NCBI General Identifier No. 16239835). SEQ ID NO:6 and SEQ ID NO:18 have identical amino acid sequences.

SEQ ID NO:19 is the amino acid sequence of abscisic acid activated protein from Medicago sativa (NCBI General Identifier No. 7431112).

SEQ ID NO:20 is the 8966 bp sequence of KS210.

SEQ ID NO:21 is the nucleotide sequence of the ALS selectable marker recombinant DNA fragment. This recombinant DNA fragment comprises a promoter operably linked to a nucleotide fragment encoding a soybean acetolactate synthase to which mutations have been introduced to make it resistant to treatment with sulfonylurea herbicides.

SEQ ID NO:22 is the amino acid sequence of the soybean herbicide-resistant ALS including mutations in subsequences B and F.

SEQ ID NO:23 is the wild type amino acid sequence of conserved ALS “subsequence B” disclosed in U.S. Pat. No. 5,013,659.

SEQ ID NO:24 is the wild type amino acid sequence of conserved ALS “subsequence F” disclosed in U.S. Pat. No. 5,013,659.

SEQ ID NO:25 is the amino acid sequence of the additional five amino acids introduced during cloning at the amino-terminus of the soybean ALS.

SEQ ID NO:26 is the 9242 bp sequence of pKS231 (ATCC Accession No. PTA-6148).

SEQ ID NO:27 is the KS210-Kpn-Hyg-sense primer used in Example 5.

SEQ ID NO:28 is the KS210-Kpn-Hyg-antisense primer used in Example 5.

SEQ ID NO:29 is the nucleotide sequence of pDN10.

SEQ ID NO:30 is the PSO33496 Not-sense primer used in Example 5.

SEQ ID NO:31 is the PSO33496 Not-antisense primer used in Example 5.

SEQ ID NO:32 is the 9861 bp sequence of pTASTE19 (PHP20765).

SEQ ID NO:33 is the PSO33498 Not-sense primer used in Example 5.

SEQ ID NO:34 is the PSO33498 Not-antisense primer used in Example 5.

SEQ ID NO:35 is the 9861 bp sequence of pTASTE20 (PHP20766).

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

All patents, patent applications, and publications cited throughout the application are incorporated by reference in their entirety.

In the context of this disclosure, a number of terms shall be utilized.

The term “aldehyde reductase” can be used interchangeably with the terms alcohol dehydrogenase (NADP+), aldehyde reductase (NADPH2), NADP-alcohol dehydrogenase, NADP-aldehyde reductase, NADP-dependent aldehyde reductase, NADPH-aldehyde reductase, NADPH-dependent aldehyde reductase, nonspecific succinic semialdehyde reductase, ALR1, low-K(m) aldehyde reductase, high-K(m) aldehyde reductase and alcohol dehydrogenase (NADP). The enzyme aldehyde reductase (EC 1.1.1.2) reversibly catalyzes the following reaction:
an alcohol+NADP+=an aldehyde+NADPH+H⁺

Some members of this group oxidize only primary alcohols as illustrated above, while others act also on secondary alcohols to form ketones.

The term enzyme “activity” refers to the ability of an enzyme to perform its normal function, i.e., to convert a substrate to a product. For example, aldehyde reductase converts aldehydes or ketones to their corresponding primary or secondary alcohols, respectively.

The term “aldehyde products” refers to any class of highly reactive organic chemical compounds characterized by the common group —CHO. The sequences of the invention are also useful to transform soybean plants to produce seeds have increase aldehyde reductase activity from which soybean products can be produced. It is believed that the increase in aldehyde reductase activity lowers the level of aldehydes and/or ketones and, thus, helps to improve flavor. Examples of volatile aldehydes and ketones which may contribute to poor flavor in soybean products include, but are not limited to hexanal, (3Z)-hexenal, (2E)-hexenal, (3Z, 6Z)-nonadienal, (2E, 6Z)-nonadienal, 2-nonanone, 2-decanone and 3-octen-2-one.

The terms “polynucleotide”, “polynucleotide sequence”, “nucleotide sequence”, “nucleic acid sequence”, and “nucleic acid fragment”/“isolated nucleic acid fragment” are used interchangeably herein. These terms encompass nucleotide fragments and the like. A nucleic acid fragment may be a polymer of RNA or DNA that is single- or double-stranded, that optionally contains synthetic, non-natural or altered nucleotide bases. A nucleic acid fragment in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of nucleic acid fragments such as deletion or insertion of one or more nucleotides that do not substantially alter the functional properties of the resulting nucleic acid fragment relative to the fragment of interest. It is therefore understood, as those skilled in the art will appreciate, that the nucleic acid fragments mentioned herein encompass more than the specific exemplary sequences.

The term “isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is made by an artificial combination of two otherwise separated segments of sequence, e.g., by chemical synthesis or by the manipulation of isolated nucleic acids by genetic engineering techniques. A “recombinant DNA construct” comprises any of the isolated polynucleotides of the present invention operably linked to at least one regulatory sequence.

As used herein, “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to nucleic acid fragments wherein changes in one or more nucleotide bases does not affect the ability of the nucleic acid fragment to mediate alteration of gene expression by gene silencing through for example antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate gene silencing or alteration of the functional properties of the resulting protein molecule. It is therefore understood that the invention encompasses more than the specific exemplary nucleotide or amino acid sequences and includes functional equivalents thereof. The terms “substantially similar” and “corresponding substantially” are used interchangeably herein.

The “Clustal V method of alignment” corresponds to the alignment method labeled Clustal V (described by Higgins and Sharp, CABIOS. 5:151-153 (1989)) and found in the Megalign program of the LASERGENE bioinformatics computing suite (DNASTAR Inc., Madison, Wis.). The “default parameters” are the parameters pre-set by the manufacturer of the program and for multiple alignments they correspond to GAP PENALTY=10 and GAP LENGTH PENALTY=10, while for pairwise alignments they are KTUPLE 1, GAP PENALTY=3, WINDOW=5 and DIAGONALS SAVED=5. After alignment of the sequences, using the Clustal V program, it is possible to obtain a “percent identity” by viewing the “sequence distances” table on the same program.

For example, it is well known in the art that antisense suppression and co-suppression of gene expression may be accomplished using nucleic acid fragments representing less than the entire coding region of a gene, and by using nucleic acid fragments that do not share 100% sequence identity with the gene to be suppressed. Moreover, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. In some instances, nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Consequently, an isolated polynucleotide comprising a nucleotide sequence of at least 30 (preferably at least 40, most preferably at least 60) contiguous nucleotides derived from a nucleotide sequence of SEQ ID NO:1, 3, 5, 7 or 9, and the complement of such nucleotide sequences may be used to affect the expression and/or function of an aldehyde reductase in a host cell. A method of using an isolated polynucleotide to affect the level of expression of a polypeptide in a host cell (eukaryotic, such as plant or yeast, prokaryotic such as bacterial) may comprise the steps of: constructing an isolated polynucleotide of the present invention or an isolated recombinant DNA construct of the present invention; introducing the isolated polynucleotide or the isolated recombinant DNA construct into a host cell; measuring the level of a polypeptide or enzyme activity in the host cell containing the isolated polynucleotide; and comparing the level of a polypeptide or enzyme activity in the host cell containing the isolated polynucleotide with the level of a polypeptide or enzyme activity in a host cell that does not contain the isolated polynucleotide.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C.

Substantially similar nucleic acid fragments of the instant invention may also be characterized by the percent identity of the amino acid sequences that they encode to the amino acid sequences disclosed herein, as determined by algorithms commonly employed by those skilled in this art. Suitable nucleic acid fragments (isolated polynucleotides of the present invention) encode polypeptides that are at least 86% identical to the amino acid sequences reported herein. More preferred nucleic acid fragments encode amino acid sequences that are at least 90% identical to the amino acid sequences reported herein. Most preferred are nucleic acid fragments that encode amino acid sequences that are at least 95% identical to the amino acid sequences reported herein. Suitable nucleic acid fragments not only have the above identities but typically encode a polypeptide having at least 50 amino acids, preferably at least 100 amino acids, more preferably at least 150 amino acids, still more preferably at least 200 amino acids, and most preferably at least 250 amino acids.

It is well understood by one skilled in the art that many levels of sequence identity are useful in identifying related polypeptide sequences. Useful examples of percent identities are 50%, 55%, 60%, 65%, 70%, 75%, 80%, 85%, 86%, 90%, or 95%, or any integer percentage from 55% to 100%. Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp, CABIOS. 5:151-153 (1989)) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein. A gene encompasses regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, and arranged in a manner different than that found in nature. A “foreign” gene refers to a gene not normally found in the host organism, that is introduced into the host organism by gene transfer. Foreign genes can comprise native genes inserted into a non-native organism, or chimeric genes. A “transgene” is a gene that has been introduced into the genome by a transformation procedure. An “allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

“Coding sequence” refers to a DNA fragment that codes for a polypeptide having a specific amino acid sequence. “Regulatory sequences” refer to nucleotides located upstream (5′ non-coding sequences), within, or downstream (3′ non-coding sequences) of a coding sequence, and which influence the transcription, RNA processing, stability, or translation of the associated coding sequence. Regulatory sequences may include, and are not limited to, promoters, translation leader sequences, introns, and polyadenylation recognition sequences.

“Promoter” refers to a region of DNA capable of controlling the expression of a coding sequence or functional RNA. The promoter sequence consists of proximal and more distal upstream elements. These upstream elements are often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”. New promoters of various types useful in plant cells are constantly being discovered; numerous examples may be found in the compilation by Okamuro, J. K., and Goldberg, R. B., Biochemistry of Plants 15:1-82 (1989).

Any promoter can be used in accordance with the method of the invention. Thus, the origin of the promoter chosen to drive expression of the coding sequence is not critical as long as it has sufficient transcriptional activity to accomplish the invention by expressing translatable mRNA for the desired nucleic acid fragments in the desired host tissue. The promoter for use in the present invention may be selected from the group consisting of a seed-specific promoter, root-specific promoter, vacuole-specific promoter, and an embryo-specific promoter.

Examples of a seed-specific promoter include, but are not limited to, the promoter for β-conglycinin (Chen et al. (1989) Dev. Genet. 10: 112-122), the napin promoter, and the phaseolin promoter. Other tissue-specific promoters that may be used to accomplish the invention include, but are not limited to, the chloroplast glutamine synthase (GS2) promoter (Edwards et al. (1990) Proc. Natl. Acad. Sci. U.S.A. 87:3459-3463), the chloroplast fructose-1,6-biophosphatase promoter (Lloyd et al. (1991) Mol. Gen. Genet. 225:209-2216), the nuclear photosynthetic (ST-LS1) promoter (Stockhaus et al. (1989) EMBO J. 8:2445-2451), the serine/threonine kinase (PAL) promoter, the glucoamylase promoter, the promoters for the Cab genes (cab6, cab-1, and cab-1 R, Yamamoto et al. (1994) Plant Cell Physiol. 35:773-778; Fejes et al. (1990) Plant Mol. Biol. 15:921-932; Lubberstedt et al. (1994) Plant Physiol. 104:997-1006; Luan et al. (1992) Plant Cell 4:971-981), the pyruvate orthophosphate dikanase promoter (Matsuoka et al. (1993) Proc. Natl. Acad. Sci. U.S.A. 90:9586-9590), the LhcB promoter (Cerdan et al. (1997) Plant Mol. Biol. 33:245-255), the PsbP promoter (Kretsch et al. (1995) Plant Mol. Biol. 28:219-229), the SUC2 sucrose H+ symporter promoter (Truernit et al. (1995) Planta 196:564-570), and the promoters for the thylakoid membrane genes (psaD, psaF, psaE, PC, FNR, atpC, atpD), etc.

A plethora of promoters is described in WO 00/18963, published on Apr. 6, 2000, the disclosure of which is hereby incorporated by reference.

The term “operably linked” refers to the association of nucleic acid fragments on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The “translation leader sequence” refers to a polynucleotide fragment located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D., Mol. Biotechnol. 3:225-236 (1995)).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989).

“RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated, yet has an effect on cellular processes. The terms “complement” and “reverse complement” are used interchangeably herein with respect to mRNA transcripts, and are meant to define the antisense RNA of the message.

The term “recombinant” refers to an artificial combination of two otherwise separated segments of sequence, e.g., by chemical synthesis or by the manipulation of isolated segments of nucleic acid fragments by genetic engineering techniques.

The terms “recombinant construct”, “expression construct”, “chimeric construct”, “construct”, “recombinant DNA construct”, and recombinant DNA fragment are used interchangeably herein and are nucleic acid fragments. A recombinant construct comprises an artificial combination of nucleic acid fragments, including and not limited to regulatory and coding sequences that are not found together in nature. For example, a chimeric construct may comprise regulatory sequences and coding sequences that are derived from different sources, or regulatory sequences and coding sequences derived from the same source, and arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The term “recombinant DNA construct” refers to a DNA construct assembled from nucleic acid fragments obtained from different sources. The types and origins of the nucleic acid fragments may be very diverse. A “recombinant DNA construct” includes and is not limited to the following combinations: a) nucleic acid fragment corresponding to a promoter operably linked to at least one nucleic acid fragment encoding a selectable marker, followed by a nucleic acid fragment corresponding to a terminator, b) a nucleic acid fragment corresponding to a promoter operably linked to a nucleic acid fragment capable of producing a stem-loop structure, and followed by a nucleic acid fragment corresponding to a terminator, and c) any combination of a) and b) above. In the stem-loop structure at least one nucleic acid fragment that is capable of suppressing expression of a native gene comprises the “loop” and is surrounded by nucleic acid fragments capable of producing a stem.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989. Transformation methods are well known to those skilled in the art and are described below.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

The term “expression”, as used herein, refers to the production of a functional end-product e.g., a mRNA or a protein (precursor or mature).

“Mature” protein refers to a post-translationally processed polypeptide; i.e., one from which any pre- or pro-peptides present in the primary translation product have been removed. “Precursor” protein refers to the primary product of translation of mRNA; i.e., with pre- and pro-peptides still present. Pre- and pro-peptides may be and are not limited to intracellular localization signals.

“Stable transformation” refers to the transfer of a nucleic acid fragment into a genome of a host organism, including nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms.

Methods for transforming dicots and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al., Plant Cell Rep. 15:653-657 (1996), McKently et al., Plant Cell Rep. 14:699-703 (1995)); papaya (Ling et al., Bio/technology 9:752-758 (1996)); and pea (Grant et al., Plant Cell Rep. 15:254-258 (1995)). For a review of other commonly used methods of plant transformation see Newell, C. A., Mol. Biotechnol. 16:53-65 (2000). One of these methods of transformation uses Agrobactedium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U.S.A. 84:3962-3966 (1987)), microinjection, or particle bombardment (McCabe et al., Bio/Technology 6:923 (1988); Christou et al., Plant Physiol. 87:671-674 (1988)).

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In.: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. The regenerated plants may be self-pollinated. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide(s) is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook et al. (1989) Molecular Cloning: A Laboratory Manual, Cold Spring Harbor Press; Maliga et al. (1995) Methods in Plant Molecular Biology, Cold Spring Harbor Press; Birren et al. (1998) Genome Analysis: Detecting Genes, 1, Cold Spring Harbor, N.Y.; Birren et al. (1998) Genome Analysis: Analyzing DNA, 2, Cold Spring Harbor, N.Y.; Plant Molecular Biology: A Laboratory Manual, eds. Clark, Springer, New York (1997)).

The terms “soy” and “soybean” are used interchangeably herein. Within the scope of the invention are soybean plants (Glycine soja or Glycine max), seeds, and plant parts obtained from such transformed plants. The terms “soybean product” and “soy product” are used interchangeably herein.

Also within the scope of the invention are soybean products derived from the transformed plants such as grain, protein products, oils, and products including such soybean products like feed and foodstuffs. Plant parts include differentiated and undifferentiated tissues, including and not limited to, roots, stems, shoots, leaves, pollen, seeds, tumor tissue, and various forms of cells and cultures such as single cells, protoplasts, embryos, and callus tissue. The plant tissue may be in plant or in organ, tissue or cell culture.

Included within the scope of this invention are soybean products that include protein isolates, protein concentrates, food products, feed products, beverages, etc.

Thus, in another aspect, this invention concerns a soybean product obtained from a soybean seed having increased aldehyde reductase activity, said soybean seed being produced by a soybean plant comprising in its genome a recombinant construct comprising a nucleotide sequence encoding a polypeptide having aldehyde reductase activity and said nucleotide sequence is operably linked to at least one regulatory sequence.

In a preferred embodiment, the nucleotide sequence encoding a polypeptide having aldehyde reductase activity comprises a nucleotide sequence selected from the group consisting of:

a) a nucleotide sequence encoding a polypeptide having aldehyde reductase activity, wherein the polypeptide has at least 86% sequence identity, based on the Clustal V method of alignment, when compared to SEQ ID NO:10;

b) a nucleotide sequence encoding a polypeptide having aldehyde reductase activity, wherein the polypeptide has at least 95% sequence identity, based on the Clustal V method of alignment, when compared to one of SEQ ID NO:2, SEQ ID NO:4, SEQ ID NO:6 or SEQ ID NO:8;

c) a nucleotide sequence encoding the amino acid sequence of SEQ ID NO:2, SEQ ID NO:4, SEQ ID NO:6, SEQ ID NO:8 or SEQ ID NO:10; and

d) a nucleotide sequence of SEQ ID NO:1, SEQ ID NO:3, SEQ ID NO:5, SEQ ID NO:7 or SEQ ID NO:9.

Thus, a soy product made from seeds of the invention includes, but is not limited to, protein isolate, protein concentrate, meal, grits, full fat and defatted flours, textured proteins, textured flours, textured concentrates and textured isolates.

Methods for obtaining such products are well known to those skilled in the art. For example soybean protein products can be obtained in a variety of ways. Conditions typically used to prepare soy protein isolates have been described by Cho et al. (U.S. Pat. No. 4,278,597) and Goodnight et al. (U.S. Pat. No. 4,072,670). Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described by Pass (U.S. Pat. No. 3,897,574) and Campbell et al. ((1985) In New Protein Foods, Ed. by Altschul and Wilcke, Academic Press, Vol. 5, Chapter 10, Seed Storage Proteins, pp 302-338).

A “soybean product” can be defined as those items produced from seeds obtained from a suitable plant which soybean product can be used in feeds, foods and/or beverages. For example, “soy protein products” can include, and are not limited to, those items listed in Table 2. “Soy protein products”.

TABLE 2
Soy Protein Products Derived from Soybean Seeds^{a}

Whole Soybean Products
Roasted Soybeans
Baked Soybeans
Soy Sprouts
Soy Milk
Specialty Soy Foods/Ingredients
Soy Milk
Tofu
Tempeh
Miso
Soy Sauce
Hydrolyzed Vegetable Protein
Whipping Protein
Processed Soy Protein Products
Full Fat and Defatted Flours
Soy Grits
Soy Hypocotyls
Soybean Meal
Soy Milk
Soy Protein Isolates
Soy Protein Concentrates
Textured Soy Proteins
Textured Flours and Concentrates
Textured Concentrates
Textured Isolates
Soy Pastes
^{a}See Soy Protein Products: Characteristics, Nutritional Aspects and Utilization (1987). Soy Protein council.

“Processing” refers to any physical and chemical methods used to obtain the products listed in Table 2 and includes, and is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale (Poult. Sci. 69:76-83 (1990)). “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size.. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass (U.S. Pat. No. 3,897,574) and Campbell et al., ((1985) In, Ed. by Altschul and Wilcke, Academic Press, Vol. 5, Chapter 10, Seed Storage Proteins, pp 302-338). “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously (Atkinson—U.S. Pat. No. 3,488,770; Horan (1985) In New Protein Foods, Ed. by Altschul and Wilcke, Academic Press, Vol. 1A, Chapter 8, pp 367-414). Moreover, conditions used during extrusion processing of complex foodstuff mixtures that include soy protein products have been described previously (Rokey (1983) Feed Manufacturing Technology III, 222-237 (1983); McCulloch—U.S. Pat. No. 4,454,804).

Also, within the scope of this invention are food, food supplements, food bars, and beverages that have incorporated therein a soybean-derived product of the invention.

It is common to refer to “food” as that matter suitable for human consumption and to “feed” as that matter suitable for consumption by livestock. A food product, then, is a product consumed by humans. For example, a food product encompasses a “dairy product” which includes, but is not limited to, a milk, a cream, a cheese, a butter, or any product derived from the milk of an animal or derived an alternative source in order to mimic those products derived from the milk of an animal. A dairy product such as margarine spreads or cheeses may also be prepared from vegetable or other means to mimic the flavor and consistency of its animal-milk-derived counterpart. Other examples of foods include, but are not limited to, a “food bar” is a type of food made in a bar shape or any other shape, “nutritional supplement” is a product that is intended to supplement the diet.

A “beverage” is a liquid, different than water, which is used for drinking. A beverage of the invention may be available as a liquid or as a powder that may be dissolved.

The foods to which the soybean-derived product of the invention can be incorporated/added include almost all foods/beverages. For example, there can be mentioned meats such as ground meats, emulsified meats, marinated meats, and meats injected with a soybean-derived product of the invention. Included may be beverages such as nutritional beverages, sports beverages, protein-fortified beverages, juices, milk, milk alternatives, and weight loss beverages. Mentioned may also be cheeses such as hard and soft cheeses, cream cheese, and cottage cheese. Included may also be frozen desserts such as ice cream, ice milk, low fat frozen desserts, and non-dairy frozen desserts. Finally, yogurts, soups, puddings, bakery products, salad dressings, spreads, and dips (such as mayonnaise and chip dips) may be included. The soybean product can be added in an amount selected to deliver a desired amount to the consumer of the food and/or beverage.

The soy product of the invention may also be incorporated into foods, such as, a cereal food product, a snack food product, a baked good product, a fried food product, a health food product, an infant formula, a beverage, a nutritional supplement, a dairy product, a pet food product, or animal feed.

A cereal food product is a food product derived from the processing of a cereal grain. A cereal grain includes any plant from the grass family that yields an edible grain (seed). The most popular grains are barley, corn, millet, oats, quinoa, rice, rye, sorghum, triticale, wheat and wild rice. Examples of a cereal food product include, but are not limited to, whole grain, crushed grain, grits, flour, bran, germ, breakfast cereals, extruded foods, pastas, and the like.

A baked good product comprises any of the cereal food products mentioned above and has been baked or processed in a manner comparable to baking, i.e., to dry or harden by subjecting to heat. Examples of a baked good product include, but are not limited to bread crumbs, baked snacks, mini-biscuits, mini-crackers, mini-cookies, and mini-pretzels.

A snack food product comprises any of the above or below described food products.

A fried food product comprises any of the above or below described food products that has been fried.

A health food product is any food product that imparts a health benefit. Many oilseed-derived food products may be considered as health foods.

As was mentioned above, the beverage can be in a liquid or in a dry powdered form.

For example, there can be mentioned non-carbonated drinks; carbonated drinks; fruit juices, fresh, frozen, canned or concentrate; still or sparkling water; flavored or plain milk drinks, etc. Adult and infant nutritional formulas are well known in the art and commercially available (e.g., Similac®, Ensure®, Jevity®, and Alimentum® from Ross Products Division, Abbott Laboratories).

Infant formulas are liquids or reconstituted powders fed to infants and young children. They serve as substitutes for human milk. Infant formulas have a special role to play in the diets of infants because they are often the only source of nutrients for infants. Although breast-feeding is still the best nourishment for infants, infant formula is a close enough second that babies not only survive but thrive. Infant formula is becoming more and more increasingly close to breast milk.

A dairy product is described above. These products include, but are not limited to, whole milk, skim milk, fermented milk products such as yogurt or sour milk, cream, butter, condensed milk, dehydrated milk, coffee whitener, ice cream, cheese, whey products, lactose, etc.

In still another aspect this invention concerns a method of producing a soybean-derived product which comprises: (a) cracking the seeds obtained from transformed plants of the invention to remove the meats from the hulls; and (b) flaking the meats obtained in step (a) to obtain the desired flake thickness.

Recombinant DNA constructs are introduced into somatic soybean embryos and transgenic soybean plants are regenerated. Various methods of transforming cells are known in the art and include Agrobacterium rhizogenes, direct delivery of DNA using PEG fusion, electroporation, microinjection (Rakoczy-Trojanowska, M., Cell. Mol. Biol. Lett. 7:849-858 (2002) or particle gun bombardment, plant virus-mediated transformation (see, U.S. Pat. No. 6,369,296 and U.S. Pat. No. 6,635,805), and liposome-mediated transformation (Rakoczy-Trojanowska, id.).

Only transformed cells are typically capable of surviving a period on selection media. Assays employed to determine the levels of aldehyde reductase activity in soybean embryos, seed chips, or bulk seed include methods known to skilled artisans and include and are not limited to assays developed for somatic embryo extracts, seed chip extracts, and bulk seed extracts. Such assays include methods such as spectrophotometric assays, SDS-polyacrylamide gel electrophoresis, and immunological assays, e.g. “western” blot or ELISA.

Assays to detect proteins may be performed by SDS-polyacrylamide gel electrophoresis or immunological assays. Assays to detect levels of substrates or products of enzymes may be performed using gas chromatography or liquid chromatography for separation and UV or visible spectrometry or mass spectrometry for detection, or the like. Determining the levels of mRNA of the enzyme of interest may be accomplished using northern-blotting or RT-PCR techniques. Once plants have been regenerated, and progeny plants homozygous for the transgene have been obtained, plants will have a stable phenotype that will be observed in similar seeds in later generations.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Preparation of cDNA Libraries and Sequencing of Entire cDNA Clones

cDNA libraries representing mRNAs from various corn, rice, soybean and wheat tissues were prepared. Some of the information included in the instant Example 1 is set forth in U.S. Pat. No. 6,274,379, granted Aug. 14, 2001. The characteristics of the libraries are described below.

TABLE 3
cDNA Libraries from Corn, Rice, Soybean and Wheat

| Library | Tissue | Clone |
|---|---|---|
| ccase-b | Corn (Zea mays L.) type II callus tissue, | ccase-b.pk0023.g2 |
| | somatic embryo formed | |
| ceb5 | Corn (Zea mays L.) amplified embryo | ceb5.pk0015.g5 |
| | 30 day | ceb5.pk0044.c8 |
| | | ceb5.pk0049.d10 |
| | | ceb5.pk0052.a4 |
| | | ceb5.pk0052.h8 |
| | | ceb5.pk0062.a4 |
| | | ceb5.pk0075.e7 |
| | | ceb5.pk0075.g12 |
| | | cen6.pk0001.g2 |
| rca1n | Rice (Oryza sativa L.) callus* | rca1n.pk022.j17 |
| sr1 | Soybean (Glycine max L.) root library | sr1.pk0003.c5:fis |
| sls2c | Soybean (Glycine max L.) infected with | sls2c.pk027.a23fis |
| | Sclerotinia sclerotiorum Mycelium | |
| wl1n | Wheat (Triticum aestivum L.) leaf 7 day | wl1n.pk0078.e5 |
| | old seedling, light grown* | |

*These libraries were normalized essentially as described in U.S. Pat. No. 5,482,845, incorporated herein by reference.

cDNA libraries representing mRNAs from various corn, rice, soybean and wheat tissues were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences or plasmid DNA was prepared from cultured bacterial cells. Amplified insert DNAs or plasmid DNAs were sequenced in dye-primer sequencing reactions to generate partial cDNA sequences (expressed sequence tags or “ESTs”; see Adams, M. D. et al., Science 252:1651 (1991)). The resulting ESTs were analyzed using a Perkin Elmer Model 377 fluorescent sequencer.

Full-insert sequence (FIS) data was generated utilizing a modified transposition protocol. Clones identified for FIS were recovered from archived glycerol stocks as single colonies, and plasmid DNAs were isolated via alkaline lysis. Isolated DNA templates were reacted with vector primed M13 forward and reverse oligonucleotides in a PCR-based sequencing reaction and loaded onto automated sequencers. Confirmation of clone identification was performed by sequence alignment to the original EST sequence from which the FIS request was made.

Confirmed templates were transposed via the Primer Island transposition kit (PE Applied Biosystems, Foster City, Calif.) which is based upon the Saccharomyces cerevisiae Ty1 transposable element (Devine and Boeke, Nucleic Acids Res. 22:3765-3772 (1994)). The in vitro transposition system places unique binding sites randomly throughout a population of large DNA molecules. The transposed DNA was then used to transform DH10B electro-competent cells (Gibco BRL/Life Technologies, Rockville, Md.) via electroporation. The transposable element contains an additional selectable marker (named DHFR; Fling and Richards, Nucleic Acids Res. 11:5147-5158 (1983)), allowing for dual selection on agar plates of only those subclones containing the integrated transposon. Multiple subclones were randomly selected from each transposition reaction, plasmid DNAs were prepared via alkaline lysis, and templates were sequenced (ABI Prism dye-terminator ReadyReaction mix) outward from the transposition event site, utilizing unique primers specific to the binding sites within the transposon.

Sequence data was collected (ABI Prism Collections) and assembled using Phred/Phrap (P. Green, University of Washington, Seattle). Phred/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies were viewed by the Consed sequence editor (D. Gordon, University of Washington, Seattle; Gordon et al., Genome Res. 8:195-202 (1998)).

### Example 2

### Characterization of cDNA Clones

cDNA clones encoding soybean aldehyde reductase were identified in the DuPont proprietary EST database. The possible function of the polypeptide encoded by each cDNA was identified by conducting BLAST (Basic Local Alignment Search Tool; Altschul, S. F., et al., J. Mol. Biol. 215:403-410 (1993)) searches of the ESTs against public databases. The searches were conducted for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The sequences were analyzed for similarity using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J., Nature Genetics 3:266-272 (1993)) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

Characterization of cDNAs encoding soybean aldehyde reductase follow. Some of the information included in the instant Example 2 is contained in U.S. Pat. No. 6,274,379, granted Aug. 14, 2001. The BLASTX search using the EST sequences from clones listed in Table 4 revealed similarity of the polypeptides encoded by the cDNAs to aldehyde reductase from Hordeum vulgare (NCBI Identifier No. GI 113595; SEQ ID NO:15), Avena fatua (NCBI Identifier No. GI 2130022; SEQ ID NO:16), Medicago sativa (NCBI Identifier No. GI 3378650; SEQ ID NO:17), Glycine max (NCBI Identifier No. GI 16239835; SEQ ID NO:18) and Medicago sativa (NCBI Identifier No. GI 7431112; SEQ ID NO:19). Shown in Table 4 are the BLASTP results obtained for the amino acid sequences of the entire aldehyde reductases encoded by the entire cDNA inserts comprising the indicated cDNA clones.

TABLE 4
BLAST Results for Sequences Encoding Polypeptides
Homologous to Aldehyde Reductase

| Clone | NCBI General | |
|---|---|---|
| (SEQ ID NO:) | Identifier No. | BLAST pLog Score |
| Contig composed of: | 113595 | 171.00 |
| ccase-b.pk0023.g2 | (SEQ ID NO: 15) | |
| ceb5.pk0015.g5 | | |
| ceb5.pk0044.c8 | | |
| ceb5.pk0049.d10 | | |
| ceb5.pk0052.a4 | | |
| ceb5.pk0052.h8 | | |
| ceb5.pk0062.a4 | | |
| ceb5.pk0075.e7 | | |
| ceb5.pk0075.g12 | | |
| cen6.pk0001.g2 | | |
| (SEQ ID NO: 2) | | |
| rca1n.pk022.j17 | 2130022 | 172.00 |
| (SEQ ID NO: 4) | (SEQ ID NO: 16) | |
| sr1.pk0003.c5:fis | 3378650 | 157.00 |
| (SEQ ID NO: 6) | (SEQ ID NO: 17) | |
| wl1n.pk0078.e5 | 3378650 | 112.00 |
| (SEQ ID NO: 8) | (SEQ ID NO: 17) | |
| sls2c.pk027.a23:fis | 16239835 | 152.00 |
| (SEQ ID NO: 10) | (SEQ ID NO: 18) | |
| sls2c.pk027.a23:fis | 7431112 | 139.00 |
| (SEQ ID NO: 10) | (SEQ ID NO: 19) | |

The nucleotide sequence corresponding to the entire cDNA insert in clone sls2c.pk027.a23:fis is shown in SEQ ID NO:9; the amino acid sequence corresponding to the translation of nucleotides 22 through 960 is shown in SEQ ID NO:10 (nucleotides 961-963 encode a stop). SEQ ID NO:6 of the instant invention is identical to SEQ ID NO:6 found in U.S. Pat. No. 6,274,379, granted Aug. 14, 2001.

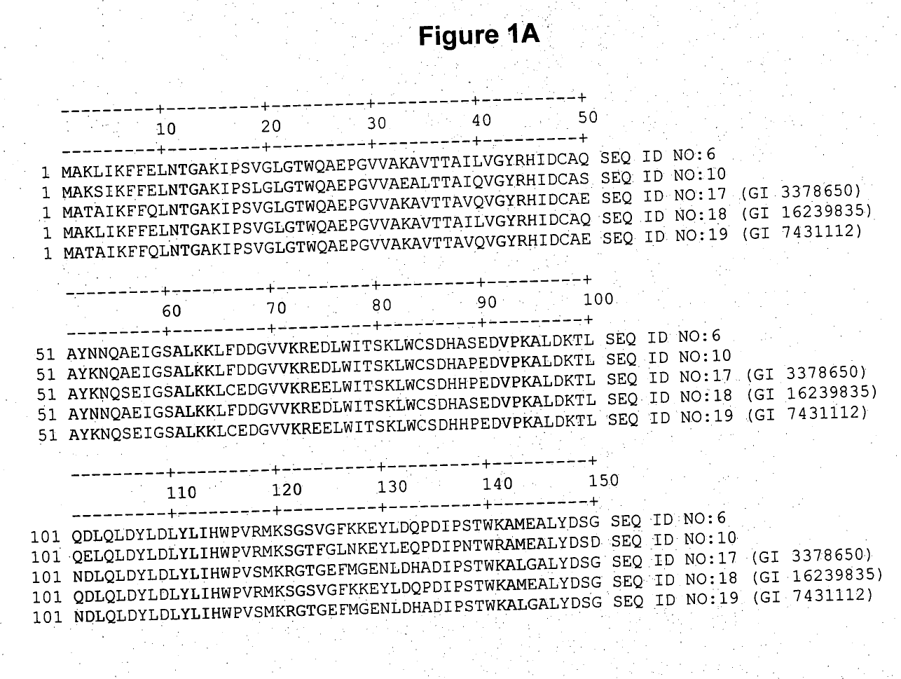

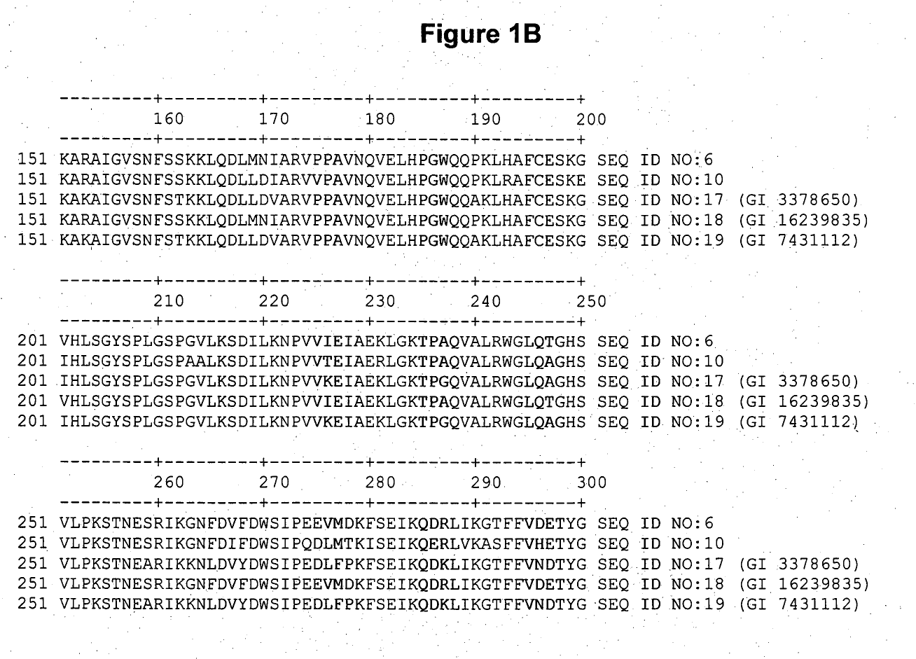

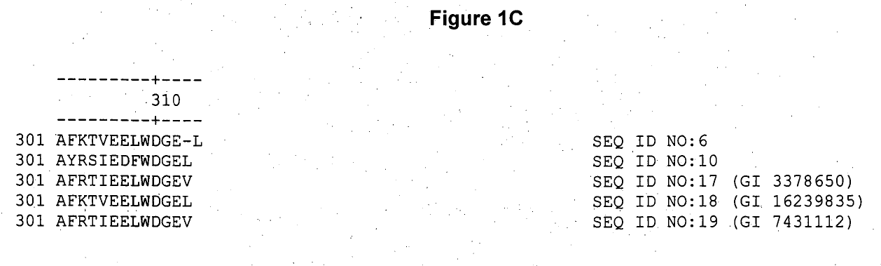

FIGS. 1A, 1B and 1C present an alignment of the amino acid sequences set forth in SEQ ID NOs:6 and 10, the amino acid sequence of abscisic acid activated (orf) protein from Medicago sativa (NCBI General Identifier No. 3378650 (SEQ ID NO:17)), the amino acid from Glycine max (NCBI General Identifier No. 16239835 (SEQ ID NO:18)) and the amino acid sequence of abscisic acid activated protein from Medicago sativa (NCBI General Identifier No. 7431112 (SEQ ID NO:19)). SEQ ID NO:6 and SEQ ID NO:18 have identical amino acid sequences. SEQ ID NO:17 and SEQ ID NO:19 have identical amino acid sequences.

The data in Table 5 presents the results obtained for the calculation of the percent identity of the amino acid sequences set forth in SEQ ID NOs:2, 4, 6, 8 and 10, with the aldehyde reductase sequences from Hordeum vulgare (NCBI General Identifier No. 113595; SEQ ID NO:15), Avena fatua (NCBI General Identifier No. 2130022; SEQ ID NO:16), Medicago sativa (NCBI General Identifier No. 3378650; SEQ ID NO:17), Glycine max (NCBI General Identifier No. 16239850; SEQ ID NO:18) and Medicago sativa (NCBI General Identifier No. 7431112; SEQ ID NO:19).

TABLE 5
Percent Identity of Deduced Amino Acid Sequences of the cDNA clones
Encoding Polypeptides Homologous to Aldehyde Reductase

| | SEQ ID | |
|---|---|---|
| Clone | NO. | Percent Identity to GI # |
| Contig composed of: | 2 | 87% to GI 113595 |
| ccase-b.pk0023.g2 | | |
| ceb5.pk0015.g5 | | |
| ceb5.pk0044.c8 | | |
| ceb5.pk0049.d10 | | |
| ceb5.pk0052.a4 | | |
| ceb5.pk0052.h8 | | |
| ceb5.pk0062.a4 | | |
| ceb5.pk0075.e7 | | |
| ceb5.pk0075.g12 | | |
| cen6.pk0001.g2 | | |
| rca1n.pk022.j17 | 4 | 90% to GI 2130022 |
| sr1.pk0003.c5:fis | 6 | 84% to GI 3378650 |
| wl1n.pk0078.e5 | 8 | 61% to GI 3378650 |
| sls2c.pk027.a23:fis | 10 | 85.4% to GI 16239835 |
| sls2c.pk027.a23:fis | 10 | 79.6% to GI 7431112 |

Sequence alignments and percent identity calculations were performed using the Megalign program of the LASARGENE bioinformatics computing suite (DNASTAR Inc., Madison, Wis.). Multiple alignment of the sequences was performed using the Clustal method of alignment (Higgins and Sharp, CABIOS. 5:151-153 (1989)) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode a substantial portion of an aldehyde reductase.

### Example 3

### Cloning and Expression of Aldehyde Reductase Clones in E. coli

Further evidence that the proteins encoded by the clones sls2c.pk027.a23:fis and sr1.pk0003.c5:fis cDNAs (SEQ ID NOs:9 and 5, respectively) are aldehyde reductases were provided by enzyme assays of the proteins obtained by expression of the clones in E. coli.

Cloning:

The polynucleotides encoding aldehyde reductase from the clones sls2c.pk027.a23:fis and sr1.pk0003.c5:fis and (SEQ ID Nos:9 and 5, respectively) were amplified using PCR. Amplification was performed using a GeneAmp PCR System 9700 machine (Applied Biosystems, Foster City, Calif.) in order to place each into Novagen's pET32-Xa/LIC expression vector. The sense primer for aldehyde reductase encoded by clone sls2c.pk027.a23:fis (SEQ ID NO:9) was 5′ GGT ATT GAG GGT CGC ATG GCC AAA TCA ATC AAA TTC 3′ (SEQ ID NO:11) while the antisense primer was 5′ AGA GGA GAG TTA GAG CCT CAG AGT TCA CCA TCC CAG 3′ (SEQ ID NO:12). The sense primer for aldehyde reductase clone sr1.pk0003.c5:fis (SEQ ID NO:5) was 5′ GGT ATT GAG GGT CGC ATG GCA AAG TTA ATA AAA TTC 3′ (SEQ ID NO:13) while the antisense primer was 5′ AGA GGA GAG TTA GAG CCT CAG AGT TCA CCA TCC CAA AG 3′ (SEQ ID NO:14). PCR was performed using Advantage High Fidelity Polymerase (Clontech) according to manufacturer's protocol. Temperature cycles for each PCR reaction were as follows: one hold of two minutes at 94° C.; 27 cycles of 94° C. for 30 seconds, 55° C. for 30 seconds, 72° C. for 90 seconds; one hold of 72° C. for 7 minutes. Each aldehyde reductases PCR product was gel-purified using the QIAquick Gel Extraction Kit (Qiagen, Valencia, Calif.), and used in cloning according to the protocol for Novagen's Xa/LIC Vector Kit. Each new vector containing the aldehyde reductase gene was transformed into BL21 (DE3) E. coli cells (Novagen) according to the manufacturer's protocol.

Protein Overexpression:

10-mL cultures were grown in a shaking incubator for eighteen hours at 37° C. in LB-Ampicillin (100 μg/mL). A 250-mL culture (LB-Amp₁₀₀) of each aldehyde reductase was inoculated with 2.5 mLs of the eighteen-hour culture, and grown at 37° C. for 1.5 hours. Protein expression was induced with IPTG (Invitrogen), at a final concentration of 1 mM. Induced cells were grown for two hours at 37° C., shaking at 200 rpm. Cells were collected by centrifugation (10000×g, 15 minutes) and stored at −80° C. until protein purification.

Purification:

Centrifuged cell pellets of the 250-mL cultures were resuspended in 5.5 mL of BugBuster Protein Extraction Reagent and 5.5 μL benzonase nuclease (both from Novagen). This mixture was incubated for twenty minutes at 25° C., shaking at 120 rpm. Centrifugation of cell mixture (16000×g, 20 minutes) was performed, and the supernatant (containing the soluble protein fraction) was saved.

The supernatant of each aldehyde reductase cell extract was purified using Sigma's HIS-Select HF Nickel Affinity Gel. A 1-mL resin bed volume was used, and purification was performed at 4° C. After loading onto the equilibrated column, soluble aldehyde reductase protein was washed with 8-mL of wash buffer (7.5 mM imidizole, 325 mM NaCl₂, 50 mM Tris-HCl pH 8.0), and eluted in 4 mL of elution buffer (250 mM imidizole, 325 mM NaCl₂, 50 mM Tris-HCl pH 8.0). After elution, a buffer exchange (into 100 mM HEPES-KOH pH 7.0; 10% (v/v) glycerol) was performed on each aldehyde reductase sample using a PD-10 column (Amersham BioSciences, Piscataway, N.J.) following the manufacturer's protocol.

Activity Assays:

The activity assay measured oxidation of NADPH on a UV500 UV-Visible double-beam Spectrometer (Unicam) by monitoring the change in A₃₄₀. The assay was performed at room temperature (approximately 23° C.) by adding all of the following reagents into a 1-mL 1-cm quartz cuvette: 500 μL of 200 mM HEPES-KOH pH 7.0, 200 μL of ddH₂O, 100 μL of 1 mM NADPH, 25-50 μL aldehyde reductase extract in a 100 μL total volume of 100 mM HEPES-KOH pH 7.0, 10% glycerol buffer. After these samples were mixed and blanked and were incubated for three minutes and a pre-substrate rate was calculated, the assay was started by addition of the substrate to the sample cuvette (solvent was added to the reference cuvette). Several different substrates were tested with each enzyme preparation. These substrates included the following: 100 μL of 100 mM glyceraldehyde, 10 μL of 100 mM glyceraldehyde, or 100 μL of 0.5% (61 mM) hexanal in v/v ethanol, or 100 μL of a carbonyl compounds mixture (ULTRA Scientific, North Kingston, R.I.; formaldehyde, acetaldehyde, propanal, butantal, pentanal, hexanal, heptanal, octanal, nonanal, decanal, cyclohexanone, and crotonaldehdye (in methanol)). The carbonyl compounds mixture contains approximately 16 μmole total aldehyde per assay. Except for the inactivity seen in one aldehyde reductase extract with hexanal as the substrate, both of the aldehyde reductase enzymes were active with each of the different substrates tested (see Table 6). As a control, an extract of E. coli proteins without the expressed aldehyde reductase enzymes was prepared and assayed the same way. No activity was observed in the presence of 100 μL of 100 mM glyceraldehyde, with NADPH as the reductant.

A Bradford assay was performed to determine protein concentrations in the extracts, and the enzymatic rates were calculated.

Table 6 presents the activity (in μmol of NADPH oxidized and were NADPH specific (i.e., no activity was observed when NADH was used as the reductant)/min⁻¹/mg protein⁻¹) obtained for the purified protein extracts from the plasmids and the source of the DNA. Enzyme activity was calculated as follows: (ΔA₃₄₀/min; after correction for pre-substrate addition rates)/(6.2×mg enzyme/mL reaction mixture).

TABLE 6
Aldehyde Reductase Activity of Expressed, Purified Proteins

| | | Other | |
|---|---|---|---|
| | | compounds | Activity |
| Source | Substrate | added to assay | μmol · min⁻¹ · mg protein⁻¹ |
| sls2c.pk027.a23:fis | 10 mM | — | 0.60 |
| (SEQ ID NO: 10) | glyceraldehyde | | |
| sls2c.pk027.a23:fis | 10 mM | — | 0.74 |
| (SEQ ID NO: 10) | glyceraldehyde | | |
| sls2c.pk027.a23:fis | 10 mM | — | 0.70 |
| (SEQ ID NO: 10) | glyceraldehyde | | |
| sls2c.pk027.a23:fis | 1 mM | — | 0.14 |
| (SEQ ID NO: 10) | glyceraldehyde | | |
| sls2c.pk027.a23:fis | 10 mM | NADH^{a} | 0.00 |
| (SEQ ID NO: 10) | glyceraldehyde | | |
| sls2c.pk027.a23:fis | 10 mM | ZnCl₂^{b} | 0.28 |
| (SEQ ID NO: 10) | glyceraldehyde | | |
| sls2c.pk027.a23:fis | 100 μL 0.5% hexanal | — | 0.00 |
| (SEQ ID NO: 10) | | | |
| sls2c.pk027.a23:fis | 100 μL aldehyde | — | 0.58 |
| (SEQ ID NO: 10) | mixture | | |
| sr1.pk0003.c5 | 10 mM | — | 1.65 |
| (SEQ ID NO: 6) | glyceraldehyde | | |
| sr1.pk0003.c5 | 10 mM | — | 1.90 |
| (SEQ ID NO: 6) | glyceraldehyde | | |
| sr1.pk0003.c5 | 10 mM | — | 2.24 |
| (SEQ ID NO: 6) | glyceraldehyde | | |
| sr1.pk0003.c5 | 1 mM | — | 0.58 |
| (SEQ ID NO: 6) | glyceraldehyde | | |
| sr1.pk0003.c5 | 10 mM | NADH^{a} | 0.00 |
| (SEQ ID NO: 6) | glyceraldehyde | | |
| sr1.pk0003.c5 | 10 mM | ZnCl₂^{b} | 0.45 |
| (SEQ ID NO: 6) | glyceraldehyde | | |
| sr1.pk0003.c5 | 100 μL 0.5% | — | 0.26 |
| (SEQ ID NO: 6) | hexanal | | |
| sr1.pk0003.c5 | 100 μL aldehyde | — | 0.55 |
| (SEQ ID NO: 6) | mixture | | |

^{a}= 100 μL of 1 mM NADH substituted for NADPH in standard assay
^{b}= 100 μL of 10 mM ZnCl₂ substituted for 100 μL ddH₂O in standard assay

### Example 4

### Expression of Chimeric Genes in Monocot Cells

A chimeric gene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (Nco I or Sma I) can be incorporated into the oligonucleotides to provide proper orientation of the DNA fragment when inserted into the digested vector pML103 as described below. Amplification is then performed in a standard PCR. The amplified DNA is then digested with restriction enzymes Nco I and Sma I and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears Accession Number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XLI-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase T DNA Sequencing Kit; U.S. Biochemical). The resulting plasmid construct would comprise a chimeric gene encoding, in the 5′ to 3′ direction, the maize 27 kD zein promoter, a cDNA fragment encoding the instant polypeptides, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al., Sci. Sin. Peking 18:659-668 (1975)). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every two to three weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al., Nature 313:810-812 (1985)) and the 3′ region of the nopaline synthase gene from the T-DNA of the Ti plasmid of Agrobacterium tumefaciens.

The particle bombardment method (Klein et al., Nature 327:70-73 (1987)) may be used to transfer genes to the callus culture cells. According to this method, gold particles (1 mm in diameter) are coated with DNA using the following technique. Ten mg of plasmid DNAs are added to 50 mL of a suspension of gold particles (60 mg per mL). Calcium chloride (50 mL of a 2.5 M solution) and permidine free base (20 mL of a 1.0 M solution) are added to the particles. The suspension is vortexed during the addition of these solutions. After 10 minutes, the tubes are briefly centrifuged (5 seconds at 15,000 rpm) and the supernatant removed. The particles are resuspended in 200 mL of absolute ethanol, centrifuged again and the supernatant removed. The ethanol rinse is performed again and the particles resuspended in a final volume of 30 mL of ethanol. An aliquot (5 mL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules, Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

### Example 5

### Transformation of Somatic Soybean (Glycine max) Embryo Cultures and Regeneration of Soybean Plants

Soybean embryogenic suspension cultures were transformed by the method of particle gun bombardment using procedures known in the art (Klein et al., Nature (London) 327:70-73 (1987); U.S. Pat. No. 4,945,050; Hazel et al., Plant Cell. Rep. 17:765-772 (1998); Samoylov et al., In Vitro Cell. Dev. Biol.—Plant 34:8-13 (1998)). In particle gun bombardment procedures it is possible to use purified 1) entire plasmid DNA or, 2) DNA fragments containing only the recombinant DNA expression cassette(s) of interest, such as those set forth below.

Construction of pDN10:

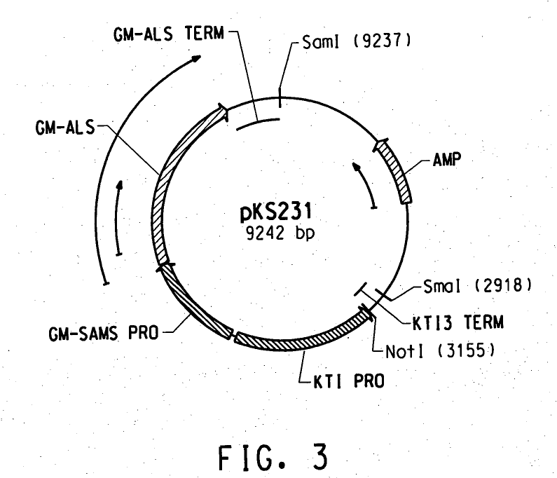

pKS231 (SEQ ID NO:26; see FIG. 3; ATCC Accession No. PTA-6148) was digested with Kpn I, run on a 1% agarose TAE gel and a 6383 bp fragment was purified using a Qiagen Gel Extraction Kit. The fragment contains a constitutive promoter directing expression of a mutant soybean acetolactate synthase (ALS) gene followed by the soybean ALS 3′ transcription terminator that is useful as a selectable marker for soybean transformation as well as a gene expression cassette that is comprised of the KTi3 promoter, a unique Not I restriction endonuclease site, and the Kti3 terminator region.

The selectable marker is comprised of a constitutive promoter directing expression of a mutant soybean acetolactate synthase (ALS) gene followed by the soybean ALS 3′ transcription terminator. The constitutive promoter used is a 1.3-Kb DNA fragment that functions as the promoter for a soybean S-adenosylmethionine synthase (SAMS) gene and is described in PCT Publication No. WO 00/37662, which published Jun. 29, 2000. The nucleotide sequence of this recombinant DNA fragment used as a selectable marker is shown in SEQ ID NO:21. The mutant soybean ALS gene encodes an enzyme that is resistant to inhibitors of ALS, such as sulfonylurea herbicides. The deduced amino acid sequence of the mutant soybean ALS present in the recombinant DNA fragment used as a selectable marker is shown in SEQ ID NO:22.

Mutant plant ALS genes encoding enzymes resistant to sulfonylurea herbicides are described in U.S. Pat. No. 5,013,659. One such mutant is the tobacco SURB-Hra gene, which encodes an herbicide-resistant ALS with two substitutions in the amino acid sequence of the protein. This tobacco herbicide-resistant ALS contains alanine instead of proline at position 191 in the conserved “subsequence B” (shown in SEQ ID NO:23) and leucine instead of tryptophan at position 568 in the conserved “subsequence F” (shown in SEQ ID NO:24) (U.S. Pat. No. 5,013,659; Lee et al., EMBO J. 7:1241-1248 (1988)).

The ALS selectable marker recombinant DNA fragment was constructed using a polynucleotide for a soybean ALS to which the two Hra-like mutations were introduced by site directed mutagenisis. Thus, this recombinant DNA fragment will translate to a soybean ALS having alanine instead of proline at position 183 and leucine instead of tryptophan at position 560. In addition, during construction of SAMS promoter-mutant ALS expression cassette, the coding region of the soybean ALS gene was extended at the 5′-end by five additional codons, resulting in five amino acids (M-P-H-N-T; shown in SEQ ID NO:25), added to the amino-terminus of the ALS protein. These extra amino acids are adjacent to and presumably removed with the transit peptide during targeting of the mutant soybean ALS protein to the plastid. A DNA fragment comprising a polynucleotide encoding the soybean ALS was digested with Kpn I, blunt ended with T4 DNA polymerase, digested with Sal I, and inserted into a plasmid containing the SAMS promoter, which had been previously digested with Nco I, and blunt ended by filling-in with Klenow DNA polymerase. For use in plant transformation experiments the ALS selectable marker recombinant DNA fragment was obtained by digesting the plasmid with restriction endonuclease Asc I and isolating the 3964 bp ALS selectable marker recombinant DNA fragment by agarose gel electrophoresis.

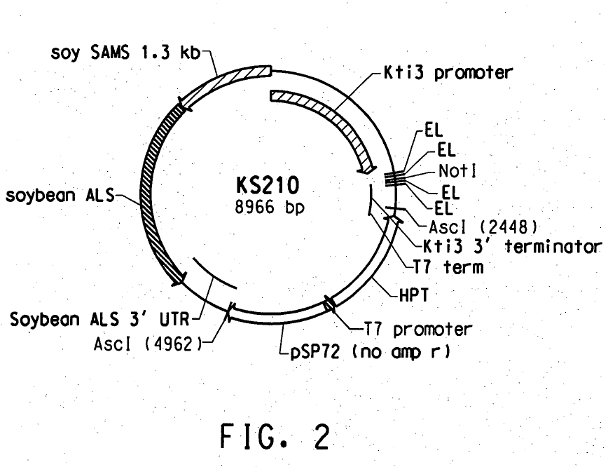

Plasmid pKS210 (SEQ ID NO:20; see FIG. 2) is derived from the commercially available cloning vector pSP72 (Promega). The beta-lactamase coding region has been replaced by a hygromycin phosphotransferase gene for use as a selectable marker in E. coli. A fragment of pKS210 containing the hygromycin phosphotransferase gene and an origin of replication is PCR amplified as follows. The primers KS210-Kpn-Hyg-sense 5′-GCCGGGGTACCGGCGCGCCCGATCATCCGGATATAGTTCC-3′ (SEQ ID NO:27) and KS210-Kpn-Hyg-antisense 5′-GCCGGGGTACCGGCGCGCCGTTCTATAGTGTCACCTAATC-3′ (SEQ ID NO:28) are used with 0.25 micrograms of pKS210 as a template with the Advantage High Fidelity 2 kit (Clontech catalog # K1914-Y) according to the manufacture's instructions. A GeneAmp PCR System 9700 machine (Applied Biosystems) machine is used with the following temperature regime: 94° C. for 15 seconds and then 28 cycles of 94° C. for 15 seconds and 67° C. for 3 minutes followed by one cycle of 67° C. for 3 minutes. The resulting 2600 bp fragment is gel purified using the Qiagen Gel Purification System, digested with Kpn I and treated with Calf alkaline phosphotase.

The two fragments described above are ligated together and transformed into E. coli. Bacterial colonies were selected and grown overnight in LB media and appropriate antibiotic selection. DNA was isolated from the resulting culture using a Qiagen Miniprep Kit according to the manufacturer's protocol and then analyzed by restriction digest. The resulting plasmid was named pDN10 (SEQ ID NO:29).

Construction of pTASTE19:

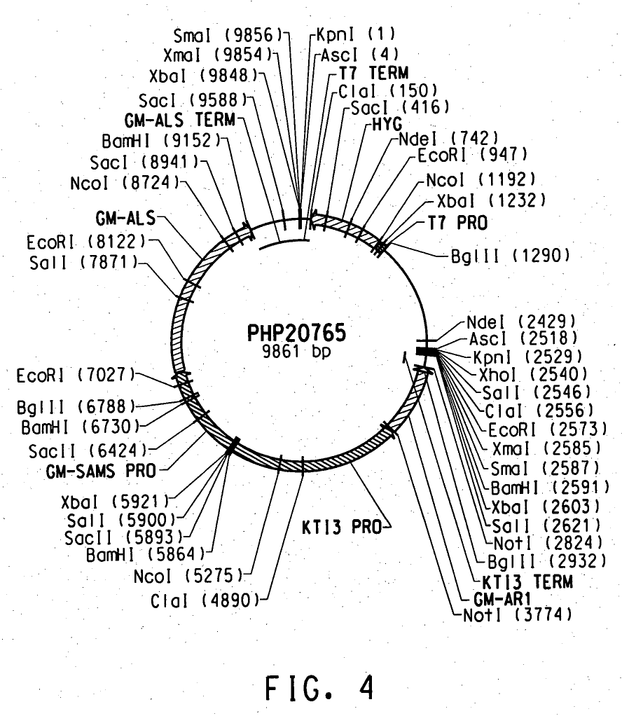

The plasmid sls2c.pk027.a23 is amplified using the primers PSO33496 Not-sense GCGGCCGCatggccaaatcaatcaaattc (SEQ ID NO:30) and PSO33496 Not-antisense GCGGCCGCtcagagttcaccatcccagaag (SEQ ID NO:31) with the Sigma Ready Mix according to the manufacturers instructions. A GeneAmp PCR System 9700 machine (Applied Biosystems) machine is used with the following temperature regime: 94° C. for 3 minutes and then 27 cycles of 94° C. for 30 seconds and 55° C. for 30 seconds and 72° C. for 90 seconds followed by one cycle of 72° C. for 7 minutes. The resulting fragment is cloned into pCR2.1 using the TOPO TA Cloning Kit (Invitrogen). This resulting plasmid is digested with Not 1, run on a TAE agarose gel and a 942 bp fragment is purified using the Qiagen Gel Purification Kit. Plasmid DN10 is digested with Not I, treated with Calf alkaline intestinal phosphotase, and gel purified. The digested plasmid and the fragment are ligated overnight and transformed into E. coli. Bacterial colonies are selected and grown overnight in LB media and appropriate antibiotic selection. DNA is isolated from the resulting culture using a Qiagen Miniprep Kit according to the manufacturer's protocol and then analyzed by restriction digest. The resulting plasmid is named pTASTE19 (SEQ ID NO:32; see FIG. 4).

Construction of pTASTE20:

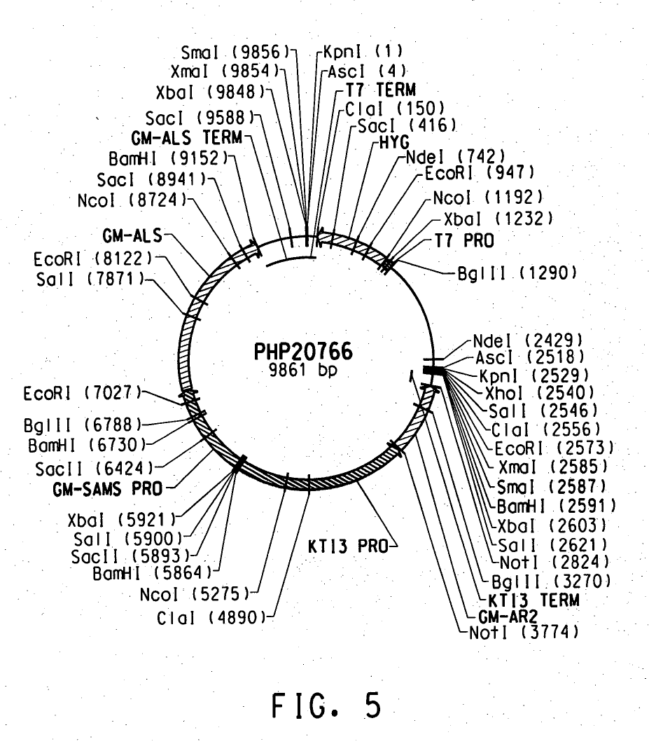

The plasmid sr1.pk003.c5 is amplified using the PSO33498 Not-sense GCGGCCGCatggcaaagttaataaaattc (SEQ ID NO:33) and PSO33498 Not-antisense GCGGCCGCtcagagttcaccatcccaaag (SEQ ID NO:34) with Advantage High Fidelity 2 kit (Clontech catalog # K1914-Y) according to the manufacturers instructions. A GeneAmp PCR System 9700 machine (Applied Biosystems) machine is used with the following temperature regime: 94° C. for 3 minutes and then 27 cycles of 94° C. for 30 seconds and 55° C. for 30 seconds and 72° C. for 90 seconds followed by one cycle of 72° C. for 7 minutes. The resulting fragment is cloned into pCR2.1 using the TOPO TA Cloning Kit (Invitrogen). This resulting plasmid is digested with Not I, run on a TAE agarose gel and a 942 bp fragment is purified using the Qiagen Gel Purification Kit. Plasmid DN10 is digested with Not I, treated with Calf alkaline intestinal phosphotase, and gel purified. The digested plasmid and the fragment are ligated overnight and transformed into E. coli. Bacterial colonies are selected and grown overnight in LB media and appropriate antibiotic selection. DNA is isolated from the resulting culture using a Qiagen Miniprep Kit according to the manufacturer's protocol and then analyzed by restriction digest. The resulting plasmid is named pTASTE20 (SEQ ID NO:35; see FIG. 5).

Transformation of Somatic Soybean (Glycine max) Embryo Cultures and Regeneration of Soybean Plants:

In the experiments that follow, the recombinant DNA fragments are isolated from the entire plasmid by Asc I digestion and gel electrophoresis before being used for bombardment. For every eight-bombardment transformation, 30 μL of solution is prepared with 3 mg of 0.6 μm gold particles and 1 to 90 picograms (pg) of DNA fragment per base pair of DNA fragment.

Stock tissue for these transformation experiments is obtained by initiation from soybean immature seeds. Secondary embryos are excised from explants after six to eight weeks on culture initiation medium. The initiation medium is an agar-solidified modified MS (Murashige and Skoog, Physiol. Plant. 15:473-497 (1962)) medium supplemented with vitamins, 2,4-D and glucose. Secondary embryos were placed in flasks in liquid culture maintenance medium and maintained for seven-nine days on a gyratory shaker at 26+/−2° C. under −80 μEm-2s-1 light intensity. The culture maintenance medium was a modified MS medium supplemented with vitamins, 2,4-D, sucrose and asparagine. Prior to bombardment, clumps of tissue were removed from the flasks and moved to an empty 60×15 mm petri dish for bombardment. Tissue was dried by blotting on Whatman #2 filter paper. Approximately 100-200 mg of tissue corresponding to 10-20 clumps (1-5 mm in size each) were used per plate of bombarded tissue.

After bombardment, tissue from each bombarded plate was divided and placed into two flasks of liquid culture maintenance medium per plate of bombarded tissue. Seven days post bombardment, the liquid medium in each flask was replaced with fresh culture maintenance medium supplemented with 100 ng/mL selective agent (selection medium). For selection of transformed soybean cells the selective agent used was a sulfonylurea (SU) compound with the chemical name, 2-chloro-N-((4-methoxy-6 methy-1,3,5-triazine-2-yl)aminocarbonyl) benzenesulfonamide (common names: DPX-W4189 and chlorsulfuron). Chlorsulfuron is the active ingredient in the DuPont sulfonylurea herbicide, GLEAN®. The selection medium containing SU was replaced every week for six-eight weeks. After the six-eight week selection period, islands of green, transformed tissue were observed growing from untransformed, necrotic embryogenic clusters. These putative transgenic events were isolated and kept in media with SU at 100 ng/mL for another two-six weeks with media changes every one-two weeks to generate new, clonally propagated, transformed embryogenic suspension cultures. Embryos spent a total of around eight-twelve weeks in SU. Suspension cultures were subcultured and maintained as clusters of immature embryos and also regenerated into whole plants by maturation and germination of individual somatic embryos.

### Example 6

### Assay of Transformed Soybean Somatic for Overexpression of Aldehyde Reductase in Seeds

Seeds from regenerated plants containing either the pTASTE19 or the pTASTE20 fragment (see Example 5) were analyzed by Western blot analysis for increased expression of the protein aldehyde reductase. The procedure was carried out as follows. Aldehyde reductase (clone ID sr1.pk003.c5 (SEQ ID NO:5)) was cloned into the pET-32 expression vector (Novagen), expressed in E. coli, and purified as described in Example 3. Centriprep 30 (Millipore) was used according to manufacturer's protocol to concentrate the purified aldehyde reductase to a concentration of 0.69 mg/mL. The purified, concentrated aldehyde reductase was sent to Covance Research Products, Inc. for development of the antibody using Elite Rabbits.

Western blotting was performed to screen individual transformed seeds for aldehyde reductase overexpression. The soybean seed protein isolation was performed by grinding the individual seeds in a 2000 Geno/Grinder (SPEX CertiPrep) for 30 seconds at 1500 strokes per minute, using a vial and ball bearing. After grinding, a total of 100 mg of ground seed was placed in a microfuge tube and 1 mL of extraction buffer (0.1% SDS+50 mM Tris-HCL, pH 8.0) was added. The samples were mixed well by vortexing, and the contents of the tube were allowed to settle at room temperature for 15 minutes. Each sample was centrifuged for 10 seconds at top speed in a microfuge, and the supernatant was discarded. The remaining, insoluble contents of the sample were mixed with 125 μL LDS Sample Buffer (Invitrogen), and heated to 95° C. for five minutes. Subsequently the samples were centrifuged for 10 seconds at top speed in a microfuge. A 1:1000 dilution of each sample was made using SDS extraction buffer, and 10 μL of each diluted sample was run on a 15-well, 10% NuPage Novex Bis-Tris Gel (Invitrogen). Gels were run at 180 volts for 40 minutes.

The XCell II Blot Module (Invitrogen) was used to transfer the seed proteins to a nitrocellulose membrane, according to the manufacturer's protocol. After blotting, nitrocellulose membranes were stained with six mL of Ponceau S stain (Sigma) by mixing at room temperature for five minutes. To destain, the blots were washed with distilled water for 10 minutes, and then washed with TBS buffer twice for 10 minutes. Blots were then blocked in 5% nonfat dry milk in TBS buffer (w/v). Blocking was carried out overnight with shaking at 4° C. Blots were washed two times for ten minutes with TBS-0.5% Tween.

The primary antibody was then mixed with the blots. The antisera received from Covance Research Products, Inc. was employed in the Western blotting protocol as the primary antibody. Those skilled in the art understand that it is preferred that the antibodies do not cross-react substantially with naturally-occurring materials that may be present in the sample (i.e., bind to other proteins present in the sample in a way that interferes with the test results). A 1:500 dilution of primary antibody was made using antibody buffer (1% nonfat dry milk (w/v) in TBS-0.5% Tween). The blot and primary antibody dilution were incubated, shaking for one hour at room temperature.

The blots were washed twice for fifteen minutes with 50 mL TBS-0.5% Tween. The secondary antibody was then added. The secondary antibody used was Pierce's ImmunoPure Peroxidase Conjugated Goat Anti-Rabbit IgG (H+L), and was prepared by diluting by 50% with glycerol (for −20° C. storage), and then diluting the resulting solution 1:25000 with antibody buffer before use. Blots were incubated with the secondary antibody for one hour at room temperature with shaking. Subsequently, blots were washed four times for fifteen minutes with 50 mL TBS-0.5% Tween.

SuperSignal West Pico Chemiluminescent Substrate (Pierce) was used to detect the presence of the HRP secondary antibody. Blots were exposed on film for one to five minutes, and the relative abundance of aldehyde reductase as shown by band intensity was noted and compared to the wild type soybean proteins that were run on the same gel. It appears that the preliminary results set forth in Table 7 were obtained using a cross-reactive antibody. Two seeds from each plant were analyzed to obtain each result. To summarize, in TASTE 19 there were 20 plants with aldehyde reductase expressed greatly above wild type levels, 34 plants with aldehyde reductase expressed moderately above wild type levels, 10 plants with aldehyde reductase expressed slightly above wild type levels and 2 plants with aldehyde reductase not expressed above wild type levels. In TASTE 20 there were 4 plants with aldehyde reductase expressed greatly above wild type levels, 30 plants with aldehyde reductase expressed moderately above wild type levels, 23 plants with aldehyde reductase expressed slightly above wild type levels and 5 plants with aldehyde reductase not expressed above wild type levels.

TABLE 7
Relative Abundance of Aldehyde Reductase

| Plant | Overexpression |
|---|---|
| Number | Results |
| TASTE 19: | |
| AFS 3697.2.1.1 | +++ |
| AFS 3697.2.1.2 | +++ |
| AFS 3697.2.3.1 | + |
| AFS 3697.2.3.2 | ++ |
| AFS 3697.2.4.1 | +++ |
| AFS 3697.2.4.2 | −−− |
| AFS 3697.3.1.1 | + |
| AFS 3697.3.1.2 | ++ |
| AFS 3697.4.1.1 | −−− |
| AFS 3697.4.1.2 | ++ |
| AFS 3697.4.2.1 | ++ |
| AFS 3697.4.2.2 | + |
| AFS 3697.5.1.1 | ++ |
| AFS 3697.5.1.2 | + |
| AFS 3697.5.2.1 | ++ |
| AFS 3697.5.3.1 | ++ |
| AFS 3697.6.2.1 | ++ |
| AFS 3697.6.2.2 | + |
| AFS 3697.7.1.1 | ++ |
| AFS 3697.7.1.2 | ++ |
| AFS 3697.7.2.1 | ++ |
| AFS 3697.7.2.2 | +++ |
| AFS 3699.2.1.1 | ++ |
| AFS 3699.2.1.2 | ++ |
| AFS 3699.5.1.3 | +++ |
| AFS 3699.5.2.1 | +++ |
| AFS 3699.5.2.2 | +++ |
| AFS 3699.5.7.1 | +++ |
| AFS 3699.5.7.2 | +++ |
| AFS 3699.5.7.3 | ++ |
| AFS 3700.1.1.1 | +++ |
| AFS 3700.1.1.2 | +++ |
| AFS 3701.1.1.1 | + |
| AFS 3701.1.1.2 | + |
| AFS 3701.2.2.1 | + |
| AFS 3701.2.2.2 | + |
| AFS 3701.2.3.1 | +++ |
| AFS 3701.2.3.2 | +++ |
| AFS 3701.3.1.1 | ++ |
| AFS 3701.3.1.2 | ++ |
| AFS 3701.3.2.1 | +++ |
| AFS 3701.3.2.2 | +++ |
| AFS 3701.3.3.1 | ++ |
| AFS 3701.3.3.2 | ++ |
| AFS 3701.4.1.1 | +++ |
| AFS 3701.4.1.2 | ++ |
| AFS 3701.4.3.1 | ++ |
| AFS 3701.4.3.2 | ++ |
| AFS 3701.4.4.1 | +++ |
| AFS 3701.4.4.2 | +++ |
| AFS 3701.5.1.1 | ++ |
| AFS 3701.5.1.2 | ++ |
| AFS 3701.5.2.1 | ++ |
| AFS 3701.5.2.2 | ++ |
| AFS 3701.7.1.1 | ++ |
| AFS 3701.7.1.2 | ++ |
| AFS 3701.7.2.1 | ++ |
| AFS 3701.7.2.2 | +++ |
| AFS 3701.7.3.1 | ++ |
| AFS 3701.7.3.2 | ++ |
| AFS 3701.7.7.1 | +++ |
| AFS 3701.7.7.2 | ++ |
| AFS 3701.7.8.1 | + |
| AFS 3701.7.8.2 | ++ |
| AFS 3701.7.9.1 | ++ |
| AFS 3701.7.9.2 | ++ |
| TASTE 20: | |
| AFS 3714.3.1.1 | ++ |
| AFS 3714.3.1.2 | ++ |
| AFS 3714.5.2.1 | +++ |
| AFS 3714.6.1.1 | ++ |
| AFS 3714.6.1.2 | ++ |
| AFS 3714.6.3.1 | ++ |
| AFS 3714.6.3.2 | ++ |
| AFS 3715.1.2.1 | ++ |
| AFS 3715.1.2.2 | ++ |
| AFS 3715.1.3.1 | ++ |
| AFS 3715.1.3.2 | ++ |
| AFS 3715.2.1.1 | ++ |
| AFS 3715.2.2.1 | −−− |
| AFS 3715.2.2.2 | + |
| AFS 3715.2.3.1 | +++ |
| AFS 3715.2.3.2 | ++ |
| AFS 3715.3.1.1 | ++ |
| AFS 3715.4.1.1 | + |
| AFS 3715.4.1.2 | + |
| AFS 3717.1.1.1 | + |
| AFS 3717.1.1.2 | −−− |
| AFS 3717.1.3.1 | ++ |
| AFS 3717.1.3.2 | + |
| AFS 3717.5.1.1 | +++ |
| AFS 3717.5.1.2 | + |
| AFS 3718.3.3.1 | ++ |
| AFS 3718.3.3.2 | ++ |
| AFS 3718.4.1.1 | ++ |
| AFS 3718.4.1.2 | ++ |
| AFS 3718.4.2.1 | ++ |
| AFS 3718.4.2.2 | + |
| AFS 3718.5.1.1 | + |
| AFS 3718.5.1.2 | + |
| AFS 3718.5.3.1 | + |
| AFS 3718.5.3.2 | ++ |
| AFS 3714.3.2.1 | ++ |
| AFS 3716.5.1.1 | + |
| AFS 3716.5.1.2 | + |
| AFS 3716.6.1.1 | −−− |
| AFS 3716.6.1.2 | + |
| AFS 3717.1.2.1 | ++ |
| AFS 3717.1.2.2 | ++ |
| AFS 3718.2.1.1 | −−− |
| AFS 3718.2.1.2 | + |
| AFS 3718.3.1.1 | + |
| AFS 3718.3.1.2 | ++ |
| AFS 3718.3.2.1 | ++ |
| AFS 3718.3.2.2 | ++ |
| AFS 3718.3.5.1 | + |
| AFS 3718.3.5.2 | ++ |
| AFS 3718.6.1.1 | + |
| AFS 3718.6.1.2 | −−− |
| AFS 3754.1.2.1 | ++ |
| AFS 3754.1.2.3 | + |
| AFS 3754.1.1.1 | ++ |
| AFS 3754.1.1.2 | +++ |
| AFS 3754.1.3.1 | ++ |
| AFS 3754.1.3.2 | + |
| AFS 3754.6.1.1 | + |
| AFS 3754.6.1.2 | + |
| AFS 3754.7.1.1 | + |
| AFS 3754.7.1.2 | + |

−−− = AR not expressed above WT levels
+ = AR expressed slightly above WT levels
++ = AR expressed moderately above WT levels
+++ = AR expressed greatly above WT levels

What is claimed is:

1. An isolated polynucleotide comprising:
(a) a nucleotide sequence encoding a polypeptide having aldehyde reductase activity, wherein the polypeptide has an amino acid sequence of at least 86% sequence identity, based on the Clustal V method of alignment, when compared to SEQ ID NO:10; or
(b) a complement of the nucleotide sequence, wherein the complement and the nucleotide sequence consist of the same number of nucleotides and are 100% complementary.

2. The polynucleotide of claim 1, wherein the amino acid sequence of the polypeptide has at least 90% sequence identity, based on the Clustal V method of alignment, when compared to SEQ ID NO:10.

3. The polynucleotide of claim 1, wherein the amino acid sequence of the polypeptide has at least 95% sequence identity, based on the Clustal V method of alignment, when compared to SEQ ID NO:10.

4. The polynucleotide of claim 1, wherein the amino acid sequence of the polypeptide comprises SEQ ID NO:10.

5. The polynucleotide of claim 1 wherein the nucleotide sequence comprises SEQ ID NO:9.

6. A vector comprising the polynucleotide of claim 1.

7. A recombinant DNA construct comprising the polynucleotide of claim 1 operably linked to at least one regulatory sequence.

8. A method for transforming a cell, comprising transforming a cell with the polynucleotide of claim 1.

9. A cell comprising the recombinant DNA construct of claim 7.

10. A method for producing a plant comprising transforming a plant cell with the polynucleotide of claim 1 and regenerating a plant from the transformed plant cell.

11. A plant comprising the recombinant DNA construct of claim 7.

12. A seed comprising the recombinant DNA construct of claim 7.

13. An isolated polypeptide having aldehyde reductase activity, wherein the polypeptide has an amino acid sequence of at least 86% sequence identity, based on the Clustal V method of alignment, when compared to SEQ ID NO:10.

14. The polypeptide of claim 13, wherein the amino acid sequence of the polypeptide has at least 90% sequence identity, based on the Clustal V method of alignment, when compared to SEQ ID NO:10.

15. The polypeptide of claim 13, wherein the amino acid sequence of the polypeptide has at least 95% sequence identity, based on the Clustal V method of alignment, when compared to SEQ ID NO:10.

16. The polypeptide of claim 13, wherein the amino acid sequence of the polypeptide comprises SEQ ID NO:10.

17. A method for isolating a polypeptide having aldehyde reductase activity comprising isolating the polypeptide from a cell or culture medium of the cell, wherein the cell comprises a recombinant DNA construct comprising the polynucleotide of claim 1 operably linked to at least one regulatory sequence.

18. A method of altering the level of expression of an aldehyde reductase in a host cell comprising: (a) transforming a host cell with the recombinant DNA construct of claim 7; and (b) growing the transformed host cell under conditions that are suitable for expression of the recombinant DNA construct wherein expression of the recombinant DNA construct results in production of altered levels of the aldehyde reductase protein in the transformed host cell.

19. A soybean product obtained from a soybean seed having increased aldehyde reductase activity, said soybean seed being produced by a soybean plant comprising in its genome a recombinant construct comprising a nucleotide sequence encoding a polypeptide having aldehyde reductase activity and said nucleotide sequence is operably linked to at least one regulatory sequence.

20. The soybean product of claim 19 wherein the nucleotide sequence encoding a polypeptide having aldehyde reductase activity comprises a nucleotide sequence selected from the group consisting of:
e) a nucleotide sequence encoding a polypeptide having aldehyde reductase activity, wherein the polypeptide has at least 86% sequence identity, based on the Clustal V method of alignment, when compared to SEQ ID NO:10;
f) a nucleotide sequence encoding a polypeptide having aldehyde reductase activity, wherein the polypeptide has at least 95% sequence identity, based on the Clustal V method of alignment, when compared to one of SEQ ID NO:2, SEQ ID NO:4, SEQ ID NO:6 or SEQ ID NO:8;
g) a nucleotide sequence encoding the amino acid sequence of SEQ ID NO:2, SEQ ID NO:4, SEQ ID NO:6, SEQ ID NO:8 or SEQ ID NO:10; and
h) a nucleotide sequence of SEQ ID NO:1, SEQ ID NO:3, SEQ ID NO:5, SEQ ID NO:7 or SEQ ID NO:9.

21. The soy product of claim 19 or 20 wherein said product is selected from the group consisting of protein isolate, protein concentrate, meal, grits, full fat and defatted flours, textured proteins, textured flours, textured concentrates, and textured isolates.

22. A food having incorporated therein the soy product of claim 19 or 20.

23. A food having incorporated therein the soy product of claim 21.

24. A beverage having incorporated therein the soy product of claim 19 or 20.

25. A beverage having incorporated therein the soy product of claim 21.

26. Feed having incorporated therein the soy product of claim 19 or 20.

27. Feed having incorporated therein the soy product of claim 21.

28. A method for increasing aldehyde reductase activity in soybean seeds, said method comprising:
a) transforming a plant part or plant tissue with a recombinant construct comprising a nucleotide sequence from at least one soybean seed aldehyde reductase gene said sequence is operably linked to at least one regulatory;
b) regenerating a transgenic plant from the plant part or plant tissue of step (a);
c) growing the transformed plant of step (b) under conditions that are suitable for the expression of the recombinant expression construct, wherein expression of said construct results in increased aldehyde reductase activity.

29. The method of claim 28 wherein the nucleotide sequence from at least one soybean seed aldehyde reductase gene comprises a nucleotide sequence selected from the group consisting of:
a) a nucleotide sequence encoding a polypeptide having aldehyde reductase activity, wherein the polypeptide has at least 86% sequence identity, based on the Clustal V method of alignment, when compared to SEQ ID NO:10;
b) a nucleotide sequence encoding a polypeptide having aldehyde reductase activity, wherein the polypeptide has at least 95% sequence identity, based on the Clustal V method of alignment, when compared to one of SEQ ID NO:2, SEQ ID NO:4, SEQ ID NO:6 or SEQ ID NO:8;
c) a nucleotide sequence encoding the amino acid sequence of SEQ ID NO:2, SEQ ID NO:4, SEQ ID NO:6, SEQ ID NO:8 or SEQ ID NO:10; and
d) a nucleotide sequence of SEQ ID NO:1, SEQ ID NO:3, SEQ ID NO:5, SEQ ID NO:7 or SEQ ID NO:9.

30. A method for producing a soy product which comprises:
(a) cracking the seeds of claim 12 to remove the meats from the hulls; and
(b) flaking the meats obtained in step (a) to obtain the desired flake thickness.

31. A method for producing a soy product which comprises:
(a) cracking seeds to remove the meats from the hulls, said seeds comprising a chimeric gene comprising an isolated polynucleotide comprising a nucleotide sequence encoding a polypeptide having aldehyde reductase activity comprises a nucleotide sequence selected from the group consisting of:
a) a nucleotide sequence encoding a polypeptide having aldehyde reductase activity, wherein the polypeptide has at least 86% sequence identity, based on the Clustal V method of alignment, when compared to SEQ ID NO:10;
b) a nucleotide sequence encoding a polypeptide having aldehyde reductase activity, wherein the polypeptide has at least 95% sequence identity, based on the Clustal V method of alignment, when compared to one of SEQ ID NO:2, SEQ ID NO:4, SEQ ID NO:6 or SEQ ID NO:8;
c) a nucleotide sequence encoding the amino acid sequence of SEQ ID NO:2, SEQ ID NO:4, SEQ ID NO:6, SEQ ID NO:8 or SEQ ID NO:10; and
d) a nucleotide sequence of SEQ ID NO:1, SEQ ID NO:3, SEQ ID NO:5, SEQ ID NO:7 or SEQ ID NO:9; and
(b) flaking the meats obtained in step (a) to obtain the desired flake thickness.


SEQUENCE LISTING (35 sequences)

SEQ ID NO: 1 - DNA, 1297 bp, Zea mays
tttttttcaagaagacaggagaagggagttgtagtgagttttaagaatggcgagtgcacaggcagtggggcaaggagaacgaggccacttcgttctgaagagcggacacaccattccggccgttggtctaggcacttggagggccggctcagataccgctcactctgttcggaccgccatcgccgaggctggatataggcacgtggacacagctgcccaatacggagtagagaaagaggtcggtagaggacttaaagctgcgatggagggcgggatcaacaggaaagatttgtttgtgacgtcgaagctatggtgcaccgagctggctcctgatagggttcggccagcactcgagaaaacactcaaggacttgcagctggattacctggatctctaccttatccactggcccttcaggctgaaagacggggcgcacatgcccccggaagccggggaggtgctggagttcgatatggaaggggtgtggagggagatggaaggcctcgtgaaagacgggctcgtcaaggatataggtgtctgcaattacacggtcgccaagctcaaccgcctgatgcggtcagcgaatgttccaccggcagtgtgccagatggaaatgcaccctgggtggaagaacgacaggatctttgaggcatgcaagaagcatgggatccatgttactgcttactctccgctgggtccgtcagagaagaacctagcgcacgacccgctcgtcgaaaaggtagccaacaaactggacaagaccccggggcaggtgctcctcaggtgggcgctccagagggggacaagcgtcattcctaaatcgaccaaggacggaaggatcaaagagaacatccaggtgttcgggtgggagatccctgaggaggacttcagggccctgtgcggcatcaaagatgagaagcgcgtgctgaccggagaggagctgttcgtgaacaagacccacgggccgtacaagagcgcgaccgaggtgtgggaccacgaggactgagcggactgccgtgccgccggatccctccactccacctgatgaaaccagaataaaggataccgacgcacctgtcagtcacctccctcccgtgccttgcgagagcggcagcctctcgcacagggaagatgctctgtgtctgagagcatgcagcctcgcacgagaaagatgcagaaggagtgtgtgtggcgcgcaatacactcctgtactgtacgatagactgaataataataaagaagaaaacgcagcagtttgccgttgcgttttcctctgtgcttgcactatcggtcgttc

SEQ ID NO: 2 - protein, 319 aa, Zea mays
MASAQAVGQGERGHFVLKSGHTIPAVGLGTWRAGSDTAHSVRTAIAEAGYRHVDTAAQYGVEKEVGRGLKAAMEGGINRKDLFVTSKLWCTELAPDRVRPALEKTLKDLQLDYLDLYLIHWPFRLKDGAHMPPEAGEVLEFDMEGVWREMEGLVKDGLVKDIGVCNYTVAKLNRLMRSANVPPAVCQMEMHPGWKNDRIFEACKKHGIHVTAYSPLGPSEKNLAHDPLVEKVANKLDKTPGQVLLRWALQRGTSVIPKSTKDGRIKENIQVFGWEIPEEDFRALCGIKDEKRVLTGEELFVNKTHGPYKSATEVWDHED

SEQ ID NO: 3 - DNA, 1277 bp, Oryza sativa
ggcacgaggaaaggtgaagatagctaaacggtgtgacaagcaaggtaatagaaaggcgcgatcatggcgagtgccaaggcgatggcgcaggatgagcatcactttgttctgaagagtggtcatgccatccctgcagttgggttaggcacttggagggccggctcagatactgctcactccgttcagacagccatcactgaggctggatacaggcatgtagatacggctgctcaatatggaatagaacaggaggtcggcaaagggcttaaagctgcgatggaagctggaatcaacaggaaagatttgtttgtgacgtcaaaaatatggtgcacaaacttggctcctgagagagttcgaccagcattaaagaacacgctgaaggatctccagttggattatatcgacctttaccttattcattggcccttccgtctaaaagatggagcacaccagcctcctgaggctggggaagtcttggagtttgacatggaggcagtatggagggaaatggagagacttgtgacagatggactggttaaggacattggtgtctgcaatttctcagttaccaagctcaacagactgttgcaatcagctaatattccacctgcagtatgccagatggaaatgcaccctggttggaagaacaataagattttcgaggcctgcaaaaaacatggaattcatgttactgcctactccccactgggttcttctgaaaagaaccttgcgcatgatccagttgtcgagaagatagccaacaagctgaacaagactccaggtcaagtgctcatcaagtgggctctccaaaggggaacaagcgttattccaaaatcaactaaagatgaaaggattaaggagaatatgcaggtgtttggatgggagatccctgaagaggacttccaggtcttgtgcggcatcaaagatgagaagcgagtcctgacaggagaggagctcttcgtgaacaagacccatgggccatacaagagtgcatctgaggtctgggataacgaggactaagctgccatgtttcacccaaagatatggcaaataatggttatgatgttggtcatgcacgaaggagttatgtgatgttctccaagcactgtgacgaaaccagctaactcagcacaagatgtactactatgtgtaaaactagtattgttctatgtgtgctcttcttcaagctttgtgtaaccagcttctcagcacaagatgtacagccagtgctgtaagataaataaaagtatcatggctttgccgttgcacttgctact

SEQ ID NO: 4 - protein, 318 aa, Oryza sativa
MASAKAMAQDEHHFVLKSGHAIPAVGLGTWRAGSDTAHSVQTAITEAGYRHVDTAAQYGIEQEVGKGLKAAMEAGINRKDLFVTSKIWCTNLAPERVRPALKNTLKDLQLDYIDLYLIHWPFRLKDGAHQPPEAGEVLEFDMEAVWREMERLVTDGLVKDIGVCNFSVTKLNRLLQSANIPPAVCQMEMHPGWKNNKIFEACKKHGIHVTAYSPLGSSEKNLAHDPVVEKIANKLNKTPGQVLIKWALQRGTSVIPKSTKDERIKENMQVFGWEIPEEDFQVLCGIKDEKRVLTGEELFVNKTHGPYKSASEVWDNED

SEQ ID NO: 5 - DNA, 1234 bp, Glycine max
cacgaggttcatcactagaagtgttggatgcatttctatttctaactaattattgtgcttattattattgttgagaaagaaaagaatggcaaagttaataaaattctttgagttgaacacaggggccaagattccttctgttgggttaggcacttggcaagctgagcctggtgttgtagccaaagctgtcaccacagccattctggttggatacaggcatattgattgtgctcaagcgtataacaatcaagcagagattggttctgctcttaagaagctttttgatgatggtgtggtgaagcgtgaggacttatggatcacctccaaactctggtgttcagatcatgcttcagaagatgtgcccaaagcattggataaaacattgcaggatttgcaacttgattaccttgacctctatctgatccactggccagtgcgcatgaaaagcggatcagttggattcaagaaggaatatctcgatcaaccggacattcccagcacatggaaagcaatggaggcactctatgactcaggcaaggcaagagccataggagttagcaatttctcttcaaaaaagcttcaagatctcatgaatatagcaagagtgcctcctgctgttaaccaagtggaattgcacccaggatggcagcagccaaagctgcatgcattctgtgaatctaaaggagttcatctgtctggatattctccactgggctcaccaggagttctcaaaagtgacattcttaagaatcctgttgtgatagagattgcagagaaattggggaagacaccggcacaagttgcccttaggtggggactgcaaacaggtcatagtgtgctgcctaagagcactaatgagtccagaatcaagggaaactttgatgtgtttgactggtctattccagaagaagtgatggataagttctctgaaattaagcaggatagactaattaagggcactttttttgttgacgagacctatggtgcctttaagaccgttgaagagctttgggatggtgaactctgagcaatatgcttcatagagatgttgacaaatgcactgctcctcaagcagattgattccgccttttacctgttaagatcttaaagctaccagctgtagtgattcaagtatgtttgagattttgagaagagctattgttcataataacacaaagtaaccgagacttatttttcatttataaaaaaaaaaaaaaaaaaaaaaaaaaaaaaa

SEQ ID NO: 6 - protein, 313 aa, Glycine max
MAKLIKFFELNTGAKIPSVGLGTWQAEPGVVAKAVTTAILVGYRHIDCAQAYNNQAEIGSALKKLFDDGVVKREDLWITSKLWCSDHASEDVPKALDKTLQDLQLDYLDLYLIHWPVRMKSGSVGFKKEYLDQPDIPSTWKAMEALYDSGKARAIGVSNFSSKKLQDLMNIARVPPAVNQVELHPGWQQPKLHAFCESKGVHLSGYSPLGSPGVLKSDILKNPVVIEIAEKLGKTPAQVALRWGLQTGHSVLPKSTNESRIKGNFDVFDWSIPEEVMDKFSEIKQDRLIKGTFFVDETYGAFKTVEELWDGEL

SEQ ID NO: 7 - DNA, 654 bp, Triticum aestivum
  misc_feature (404)..(404): n = a, c, g or t
attaaagatggctgaatcctttgttctcagtaccggctcgaggatcccatcggttgggcttggcgtatggcaaatacaacctgacgctgccaacgacgccatctacgctgctgtcaaggctgggtatcggcatattgactgtgcagcagcatacaacaatgaggaggaggtgggcctggctttgaagaaattatttgaagatggtgtggttaagcgtgatgatttgtttatcacctctaagctatgggctgctaatcatgcacctgaagatgtggaagagggaatcgacaccacacttcaagatttgcagcttgactacttgggacttgtacctcatccatggtccaatccgcatcaaaaaaaggaactaacacgatgaccctgaaaacttctccctacagatnccctgctacatgggcagcgatggagaattatacnatccggcaaaactcntgcaatccgcgtgatnactcncttgtaagaactcatgattgctnccgtncacaatgccccacatcaacaagtnantncaccggttggnacaagaaactcggaacttcaatcaaggngtcacttcgatccgcttagcactgatcctgataaggcattctaaaccatgggccgtcaaaatnnaaaacncatcccacggacang

SEQ ID NO: 8 - protein, 308 aa, Triticum aestivum
FVLSTGSRIPSVGLGVWQIQPDAANDAIYAAVKAGYRHIDCAAAYNNEEEVGLALKKLFEDGVVKRDDLFITSKLWAANHAPEDVEEGIDTTLQDLQLDYLDLYLIHGPIRIKKGTSTMTPENFLPTDIPATWAAMEKLYDSGKARAIGVSNFSCKKLHDLLAVARVPPAVNQVECHPVWQQDKLRKLCQSNGVHLSAFSPLGSPGSPWINGPSVLKNPIVVSVADKLQKTPAQVALRWGIQMGHSVLPKSANESRIKENIDIFGWSIPEDLMAKFSEIKQVRLLTAEFVVHPQAGYNTLEDFWDGEI

SEQ ID NO: 9 - DNA, 1447 bp, Glycine max
cacgagattgagagagacaaaatggccaaatcaatcaaattctttgagttgaacacaggggccaagattccttctcttgggttagggacttggcaagctgaacctggtgttgtagctgaagctctcaccacagccattcaggttggatacaggcatattgactgtgctagtgcgtataagaatcaagcagagattggttctgctcttaagaagctttttgatgatggtgtggtgaagcgtgaggacttatggatcacttccaaactctggtgttcagatcatgcaccagaagatgtgccaaaagcattggataaaacattacaggaattgcaacttgactatcttgacctctatctgatccactggccagtccgcatgaaaagtggaacatttggactcaataaagaatatcttgagcaaccagacattcccaatacatggagagcaatggaggcactctatgactcagacaaggctagagccataggagtgagcaatttctcttcaaagaagcttcaagatttgttagatatagcaagagtggttcctgctgttaaccaagtggaattgcacccaggatggcagcagccaaagttgcgcgcattttgtgaatctaaagaaattcatctatctggatattctccactaggttcaccagcagctctcaaaagtgatattcttaagaatcctgttgtcacagagattgcagagagattggggaagacaccagcacaagttgcccttcgatggggcttgcaagcaggtcatagtgtgcttcctaagagcactaatgagtccagaatcaagggaaattttgatatctttgactggtctattcctcaagatttgatgaccaagatctctgaaattaagcaggaaaggctagttaaggccagtttttttgttcacgagacctatggtgcctataggagcattgaagacttctgggatggtgaactctgagcaatatgcatctttgagacgttgaaaaatacattgctcctagagcaaatttattctgccttatacccactagttgtagtgattcaagtgagattttgagaagtgttactattgatggtaacaccgaataagtagccaagagttatttttcatttaatgattgtatcttttattcaatataataaagtgagttgagtgaatttgacaggtccactgactaaaaaaaaaaaaaaaaaactcgagactacgttgttgaggtgttcaattttttttttttttttttattattctgttttcgctgtttttagtttagcacaggatgcaggacaaggacagggtcagtcagttattgttctctgcattttgttcccaaatgaaatgtctatgactttcttcgtcaagttctagagtaacaaattttattgttgccttgcttgttattatactaagaataaaaatgttctcttatgggccaaaaaaaaaa

SEQ ID NO: 10 - protein, 313 aa, Glycine max
MAKSIKFFELNTGAKIPSLGLGTWQAEPGVVAEALTTAIQVGYRHIDCASAYKNQAEIGSALKKLFDDGVVKREDLWITSKLWCSDHAPEDVPKALDKTLQELQLDYLDLYLIHWPVRMKSGTFGLNKEYLEQPDIPNTWRAMEALYDSDKARAIGVSNFSSKKLQDLLDIARVVPAVNQVELHPGWQQPKLRAFCESKEIHLSGYSPLGSPAALKSDILKNPVVTEIAERLGKTPAQVALRWGLQAGHSVLPKSTNESRIKGNFDIFDWSIPQDLMTKISEIKQERLVKASFFVHETYGAYRSIEDFWDGEL

SEQ ID NO: 11 - DNA, 36 bp, Artificial Sequence
  primer
ggtattgagggtcgcatggccaaatcaatcaaattc

SEQ ID NO: 12 - DNA, 36 bp, Artificial Sequence
  primer
agaggagagttagagcctcagagttcaccatcccag

SEQ ID NO: 13 - DNA, 36 bp, Artificial Sequence
  primer
ggtattgagggtcgcatggcaaagttaataaaattc

SEQ ID NO: 14 - DNA, 38 bp, Artificial Sequence
  primer
agaggagagttagagcctcagagttcaccatcccaaag

SEQ ID NO: 15 - protein, 320 aa, Hordeum vulgare
MASAKATMGQGEQDHFVLKSGHAMPAVGLGTWRAGSDTAHSVRTAITEAGYRHVDTAAEYGVEKEVGKGLKAAMEAGIDRKDLFVTSKIWCTNLAPERVRPALENTLKDLQLDYIDLYHIHWPFRLKDGAHMPPEAGEVLEFDMEGVWKEMENLVKDGLVKDIGVCNYTVTKLNRLLRSAKIPPAVCQMEMHPGWKNDKIFEACKKHGIHVTAYSPLGSSEKNLAHDPVVEKVANKLNKTPGQVLIKWALQRGTSVIPKSSKDERIKENIQVFGWEIPEEDFKVLCSIKDEKRVLTGEELFVNKTHGPYRSAADVWDHEN

SEQ ID NO: 16 - protein, 319 aa, Avena fatua
MASAKAMGQGEQDRFVLKSGHAIPAVGLGTWRAGSDTAHSVQTAITEAGYRHVDTAAQYGIEKEVDKGLKAAMEAGIDRKDLFVTSKIWRTNLAPERARPALENTLKDLQLDYIDLYLIHWPFRLKDGAHQPPEAGEVLEFDMEGVWKEMEKLVKDGLVKDIDVCNFTVTKLNRLLRSANIPPAVCQMEMHPGWKNDKIFEACKKHGIHVTAYSPLGSSEKNLVHDPVVEKVANKLNKTPGQVLIKWALQRGTSVIPKSSKDERIKENIQAFGWEIPEDDFQVLCSIKDEKRVLTGEELFVNKTHGPYKSASEVWDHEN

SEQ ID NO: 17 - protein, 313 aa, Medicago sativa
MATAIKFFQLNTGAKIPSVGLGTWQAEPGVVAKAVTTAVQVGYRHIDCAEAYKNQSEIGSALKKLCEDGVVKREELWITSKLWCSDHHPEDVPKALDKTLNDLQLDYLDLYLIHWPVSMKRGTGEFMGENLDHADIPSTWKALGALYDSGKAKAIGVSNFSTKKLQDLLDVARVPPAVNQVELHPGWQQAKLHAFCESKGIHLSGYSPLGSPGVLKSDILKNPVVKEIAEKLGKTPGQVALRWGLQAGHSVLPKSTNEARIKKNLDVYDWSIPEDLFPKFSEIKQDKLIKGTFFVNDTYGAFRTIEELWDGEV

SEQ ID NO: 18 - protein, 313 aa, Glycine max; identical to SEQ ID NO: 6

SEQ ID NO: 19 - protein, 313 aa, Medicago sativa; identical to SEQ ID NO: 17

SEQ ID NO: 20 - DNA, 8966 bp, Artificial Sequence
  KS210
gatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcg

SEQ ID NO: 21 - DNA, 3964 bp, Artificial Sequence
  nucleotide sequence of ALS selectable marker
ggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 22 - protein, 656 aa, Glycine max
MPHNTMAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVARRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 23 - protein, 4 aa, Artificial Sequence
  conserved ALS “subsequence B”
GQVP

SEQ ID NO: 24 - protein, 10 aa, Artificial Sequence
  conserved ALS “subsequence F”
GMVXQWEDRF

SEQ ID NO: 25 - protein, 5 aa, Artificial Sequence
  additional five amin acids introduced
MPHNT

SEQ ID NO: 26 - DNA, 9242 bp, Artificial Sequence
  pKS231 (ATCC Accession No. PTA-6148)
ccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 27 - DNA, 40 bp, Artificial Sequence
  primer
gccggggtaccggcgcgcccgatcatccggatatagttcc

SEQ ID NO: 28 - DNA, 40 bp, Artificial Sequence
  primer
gccggggtaccggcgcgccgttctatagtgtcacctaatc

SEQ ID NO: 29 - DNA, 8911 bp, Artificial Sequence
  pDN10
ggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcga

SEQ ID NO: 30 - DNA, 29 bp, Artificial Sequence
  primer
gcggccgcatggccaaatcaatcaaattc

SEQ ID NO: 31 - DNA, 30 bp, Artificial Sequence
  primer
gcggccgctcagagttcaccatcccagaag

SEQ ID NO: 32 - DNA, 9861 bp, Artificial Sequence
  pTASTE19
cggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgctcagagttcaccatcccagaagtcttcaatgctcctataggcaccataggtctcgtgaacaaaaaaactggccttaactagcctttcctgcttaatttcagagatcttggtcatcaaatcttgaggaatagaccagtcaaagatatcaaaatttcccttgattctggactcattagtgctcttaggaagcacactatgacctgcttgcaagccccatcgaagggcaacttgtgctggtgtcttccccaatctctctgcaatctctgtgacaacaggattcttaagaatatcacttttgagagctgctggtgaacctagtggagaatatccagatagatgaatttctttagattcacaaaatgcgcgcaactttggctgctgccatcctgggtgcaattccacttggttaacagcaggaaccactcttgctatatctaacaaatcttgaagcttctttgaagagaaattgctcactcctatggctctagccttgtctgagtcatagagtgcctccattgctctccatgtattgggaatgtctggttgctcaagatattctttattgagtccaaatgttccacttttcatgcggactggccagtggatcagatagaggtcaagatagtcaagttgcaattcctgtaatgttttatccaatgcttttggcacatcttctggtgcatgatctgaacaccagagtttggaagtgatccataagtcctcacgcttcaccacaccatcatcaaaaagcttcttaagagcagaaccaatctctgcttgattcttatacgcactagcacagtcaatatgcctgtatccaacctgaatggctgtggtgagagcttcagctacaacaccaggttcagcttgccaagtccctaacccaagagaaggaatcttggcccctgtgttcaactcaaagaatttgattgatttggccatgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 33 - DNA, 28 bp, Artificial Sequence
  primer
gcggccgcatggcaaagttaataaaatt

SEQ ID NO: 34 - DNA, 29 bp, Artificial Sequence
  primer
gcggccgctcagagttcaccatcccaaag

SEQ ID NO: 35 - DNA, 9861 bp, Artificial Sequence
  pTASTE20
cggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgctcagagttcaccatcccaaagctcttcaacggtcttaaaggcaccataggtctcgtcaacaaaaaaagtgcccttaattagtctatcctgcttaatttcagagaacttatccatcacttcttctggaatagaccagtcaaacacatcaaagtttcccttgattctggactcattagtgctcttaggcagcacactatgacctgtttgcagtccccacctaagggcaacttgtgccggtgtcttccccaatttctctgcaatctctatcacaacaggattcttaagaatgtcacttttgagaactcctggtgagcccagtggagaatatccagacagatgaactcctttagattcacagaatgcatgcagctttggctgctgccatcctgggtgcaattccacttggttaacagcaggaggcactcttgctatattcatgagatcttgaagcttttttgaagagaaattgctaactcctatggctcttgccttgcctgagtcatagagtgcctccattgctttccatgtgctgggaatgtccggttgatcgagatattccttcttgaatccaactgatccgcttttcatgcgcactggccagtggatcagatagaggtcaaggtaatcaagttgcaaatcctgcaatgttttatccaatgctttgggcacatcttctgaagcatgatctgaacaccagagtttggaggtgatccataagtcctcacgcttcaccacaccatcatcaaaaagcttcttaagagcagaaccaatctctgcttgattgttatacgcttgagcacaatcaatatgcctgtatccaaccagaatggctgtggtgacagctttggctacaacaccaggctcagcttgccaagtgcctaacccaacagaaggaatcttggcccctgtgttcaactcaaagaattttattaactttgccatgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac